(19) United States
(12) Patent Application Publication
(10) Pub. No.: US 2026/0263181 A1
(43) Pub. Date: Sep. 10, 2026

(54) ENHANCED VISIBILITY OF ROBOT WHILE SURGEON IMMERSED IN VIEWER

(71) Applicant: Auris Health, Inc., Santa Clara, CA (US)

(72) Inventors: Jacob Childs, Huntington Beach, CA (US); Melissa Stachulski, Novi, MI (US)

(21) Appl. No.: 19/666,746

(22) Filed: May 4, 2026

Related U.S. Application Data
(63) Continuation of application No. PCT/IB2024/060979, filed on Nov. 6, 2024.
(60) Provisional application No. 63/596,499, filed on Nov. 6, 2023.

(51) Int. Cl. A61B 34/37; A61B 17/00; A61B 34/30

(52) U.S. Cl. CPC A61B 34/37; A61B 17/00; A61B 2017/00199; A61B 2017/00477; A61B 2034/301; A61B 2034/305

(57) ABSTRACT

A surgical robotic system comprises a plurality of robotic arms comprising an active robotic arm and an inactive robotic arm, and a console communicatively coupled to the robotic arms. The console comprises a first haptic interface device (HID) and a second HID, and a display configured to display an interactive menu comprising an active zone element and an inactive zone element, indicate, in the active zone element, data associated with a first instrument coupled to the active robotic arm based on whether the active robotic arm is assigned to be controlled by the first HID or the second HID, and indicate, in the inactive zone element, data associated with a second instrument coupled to the inactive robotic arm, wherein the inactive robotic arm is disengaged from the first HID and the second HID.

### PRIORITY

This application is a continuation of International Application No. PCT/IB2024/060979, filed Nov. 6, 2024, entitled “ENHANCED VISIBILITY OF ROBOT WHILE SURGEON IMMERSED IN VIEWER,” which claims priority to U.S. Provisional Application No. 63/596,499, filed Nov. 6, 2023, entitled “ENHANCED VISIBILITY OF ROBOT WHILE SURGEON IMMERSED IN VIEWER,” the disclosure of which is incorporated by reference herein, in its entirety.

### TECHNICAL FIELD

The systems and methods disclosed herein are directed to devices and methods for indicating information regarding instruments for performing procedures, and more particularly to robotic systems that display information regarding the instruments.

### BACKGROUND

A robotic system may be used to perform various tasks and procedures. Robotic systems may be used throughout a variety of different industries, such as manufacturing, automotive, healthcare, construction, etc. For example, in the healthcare industry, robotic surgical systems have been used to perform a vast array of medical procedures, including both minimally invasive procedures (e.g., laparoscopic procedures) and non-invasive procedures, (e.g., endoscopic procedures). In general, robotic systems may include robotic arms configured to control the movement of tools or instruments attached to the robotic arms and a console through which a user may control the movements of the robotic arms and/or tools.

### SUMMARY

In an embodiment, a surgical robotic system is disclosed. The surgical robotic system comprises a plurality of robotic arms comprising an active robotic arm and an inactive robotic arm, and a console communicatively coupled to the robotic arms. The console comprises a first haptic interface device (HID) and a second HID, and a display configured to display an interactive menu comprising an active zone element and an inactive zone element, indicate, in the active zone element, data associated with a first instrument coupled to the active robotic arm based on whether the active robotic arm is assigned to be controlled by the first HID or the second HID, and indicate, in the inactive zone element, data associated with a second instrument coupled to the inactive robotic arm, wherein the inactive robotic arm is disengaged from the first HID and the second HID.

In another embodiment, a method performed by a surgical robotic system is disclosed. The method comprises displaying, at a console of the surgical robotic system, an interactive menu comprising an active zone element and an inactive zone element, wherein the active zone element comprises a left segment, a center segment, and a right segment, indicating, in the left segment of the active zone element, a first instrument coupled to a first robotic arm of the surgical robotic system and controlled by a left haptic interface device (HID) of the console, indicating, in the right segment of the active zone element, a second instrument coupled to a second robotic arm of the surgical robotic system and controlled by a right HID of the console, indicating, in the center segment of the active zone element, data associated with a camera coupled to a third robotic arm of the surgical robotic system, and indicating, in the inactive zone element, a third instrument coupled to a fourth robotic arm, wherein the right HID is engaged to control the fourth robotic arm when an instruction is received to switch control of the right HID from the second instrument to the third instrument.

In yet another embodiment, a surgical robotic system is disclosed. The surgical robotic system comprises a plurality of robotic arms and a console communicatively coupled to the robotic arms. The plurality of robotic arms comprise a first robotic arm coupled to a first instrument, a second robotic arm coupled to a second instrument, and a third robotic arm coupled to a third instrument. The console comprises a first haptic interface device (HID) configured to control the first robotic arm, a second HID configured to control either the first robotic arm or the second robotic arm, a display configured to display an interactive menu comprising an active zone element and an inactive zone element, wherein the active zone element comprises a first segment, a center segment, and a second segment, wherein the first segment indicates the first instrument as being controlled by the first HID, wherein the second segment indicates the second instrument as being controlled by the second HID, and wherein the inactive zone element indicates the third instrument as being disengaged from the first HID and the second HID, and a processor coupled to the display and configured to receive an instruction to switch control of the second HID from the second instrument to the third instrument, wherein, in response to the instruction, the display is configured to indicate the third instrument in the second segment and the second instrument in the inactive zone element.

In yet another embodiment, a method performed by a surgical robotic system is disclosed. The method comprises displaying, at a console of the surgical robotic system, an interactive menu comprising an active zone element and an inactive zone element, indicating, in the active zone element, data associated with one or more instruments respectively coupled to one or more active robotic arms in the surgical robotic system based on whether one or more active robotic arms are assigned to be controlled by a first haptic interface device (HID) or a second HID of the console, wherein the one or more active robotic arms are actively controlled by at least one of the first HID or the second HID, and indicating, in the inactive zone element, data associated with an instrument coupled to one or more inactive robotic arms in the surgical robotic system, wherein the one or more inactive robotic arms are disengaged from the first HID and the second HID.

In yet another embodiment, a non-transitory, computer-readable medium is disclosed. The non-transitory, computer-readable medium stores instructions which, when executed by a processor of a surgical robotic system, cause the processor to display, at a console of the surgical robotic system, an interactive menu comprising an active zone element and an inactive zone element, indicate, in the active zone element, data associated with one or more instruments respectively coupled to one or more active robotic arms in the surgical robotic system based on whether one or more active robotic arms are assigned to be controlled by a first haptic interface device (HID) or a second HID of the console, wherein the one or more active robotic arms are actively controlled by at least one of the first HID or the second HID, and indicate, in the inactive zone element, data associated with an instrument coupled to one or more inactive robotic arms in the surgical robotic system, wherein the one or more inactive robotic arms are disengaged from the first HID and the second HID.

Note that the various examples described above can be combined with any other examples described herein. The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the inventive subject matter.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates the display of an interactive menu at a console of a robotic system according to various embodiments of the disclosure.

FIGS. 2A and 2B illustrate the interactive menu of FIG. 1 according to various of embodiments of the disclosure.

FIG. 3 illustrates the interactive menu of FIG. 1 according to various embodiments of the disclosure.

FIG. 4 illustrates the interactive menu of FIGS. 1-3 dynamically changing based on user input received from the console according to various embodiments of the disclosure.

FIGS. 5A and 5B illustrate the interactive menu of FIGS. 1-3 dynamically changing based on user input received from the console according to various embodiments of the disclosure.

FIGS. 6A and 6B illustrate the interactive menu of FIGS. 1-3 dynamically changing based on user input received from the console according to various embodiments of the disclosure.

FIG. 7 illustrates the interactive menu of FIGS. 1-3 dynamically changing based on user input received from the console according to various embodiments of the disclosure.

FIGS. 8A, 8B, and 8C illustrate the interactive menu of FIGS. 1-3 dynamically changing based on user input received from the console according to various embodiments of the disclosure.

FIGS. 9A and 9B illustrate examples of status indicators that may be displayed in the interactive menu of FIGS. 1-3 according to various embodiments of the disclosures.

FIGS. 10A, 10B, 10C, and 10D illustrate examples of the interactive menu including the status indicators of FIG. 9 according to various embodiments of the disclosure.

FIG. 11 illustrates the display of a table indicator relative to the interactive menu of FIGS. 1-3 according to various embodiments of the disclosure.

FIG. 12 is a flowchart illustrating a first method according to an embodiment of the disclosure.

FIG. 13 is a flowchart illustrating a second method according to an embodiment of the disclosure.

FIG. 14 is a flowchart illustrating a third method according to an embodiment of the disclosure.

FIG. 15 is a schematic diagram illustrating electronic components of a surgical robotic system in accordance with some embodiments.

### DETAILED DESCRIPTION

### A. Robotic Systems

Robotic systems may include various components that work together to perform specific tasks. While the specific design and functionality of the robotic system may vary based on the industry and application, some examples of robotic systems may include one or more robotic arms communicatively coupled to a console. The console may be used to manipulate the robotic arms and instruments detachably attached to the robotic arms. The robotic arm may include one or more joints and links interconnecting the joints. The robotic arm may also include an end effector (e.g., gripper, tool, instrument, etc.) positioned at or detachably attached to the robotic arm, such that the end effector interacts with an environment or subject.

The console of the robotic system may essentially function as a control center or interface, allowing operators or users to interact with and control the robot. The console may serve as a point of command and feedback of the robotic system, providing mechanisms for users to input instructions, monitor the status of various components of the robotic system, and receive information regarding the components and environment of the robotic system. For example, the console may include a display device configured to display an image captured by a camera of the robotic system, data describing the components of the robotic system, settings and statuses of the components of the robotic system, data related to the control of the robotic system, and other data describing the robotic system. The console may also include one or more input devices configured to control the robotic arms/end effectors of the system. The input devices, may include, for example, buttons, switches, touch sensitive surfaces, gimbals, haptic interface devices (HIDs), foot pedals, etc., positioned at various areas of the console (e.g., near the hands of the user or near the feet of the user). The user of the console may actuate or interface with the input devices to control the robotic arms/end effectors, manipulate the information being displayed at the console, adjust the settings of the console, etc. As should be appreciated, robotic systems may include many other components aside from the robotic arms and the console, such as, for example, processing hardware, sensors, power sources, safety systems, programming interfaces, memories, etc.

For illustrative purposes, various embodiments may be described herein with respect to a surgical robotic system used in the medical industry. However, it should be appreciated that the robotic system may apply to numerous other industries, and the robotic system described herein should not be limited to solely the surgical robotic system.

To this end, an example surgical robotic system may include multiple robotic arms, the movement of which may be controlled by HIDs located on a console of the surgical robotic system. The robotic arms may be positioned in a manner that facilitates performance of a procedure on a patient, and a medical provider may manipulate the HIDs to control movement of the end effectors (e.g., which may include medical instruments, cameras, etc.). The end effectors may interact with or be positioned within the body of the patient, and as such, the HIDs may be used to control movement of the end effectors within or on a patient during a procedure. At least one of the instruments may include a camera, which may be used to capture a live image of the patient anatomy during the procedure. The image may also show the movement of the end effectors with respect to the patient anatomy. The display device of the console may display the image in real-time, providing a visual feedback to the medical provider while performing the procedure, which may be used to make informed decisions during the procedure.

### B. Display Device of the Robotic System

In some cases, the display device of the console may be embodied as a headset device, in which the structure of the display device essentially envelops an area around the eyes of the user to create a sense of immersion with the image and the data displayed by the display device. When the user is immersed, the user may insert his or her head into the display device in such a manner so as to block out ambient light. In this way, the user is enabled to focus on the image (e.g., the patient anatomy) and the movement of the instruments while performing various tasks (e.g., the procedure on the patient), which may be shown in the display device. The user may perform these tasks without being distracted by any movement or occurrences happening outside of immersion. For example, immersion at the display device reduces the possibility of the medical provider being distracted during a procedure, which might otherwise result in harm or injury to the patient.

In some cases, immersion at the display device limits the amount of information that the user has access to while performing tasks using the robotic arms. For example, the display device may be limited to showing an image of a patient received by a camera scoping the patient. Sometimes, basic details regarding the instruments being used by the robotic system may also be displayed. In this way, the information displayed at the display device may generally be minimized to, again, reduce the risk of distracting the user from the primary focus of the procedure.

However, detailed information regarding the instruments may be crucial to making informed decisions during the procedure. For example, the information displayed at the display device may not indicate a handedness of the system in an easy to understand format that is also spatially conservative (i.e., minimizes display size at the display device). The term “handedness” may refer to the assignment of control of an instrument (and thus, the associated robotic arm to which the instrument is coupled) to a specific input device at the console. For example, the handedness of the robotic system may indicate that a first instrument attached to a first robotic arm is controlled by a left-side HID (i.e., input device) of the console, and a second instrument attached to a second robotic arm is controlled by a right-side HID of the console. In addition, the information displayed at the display device may not indicate a difference between the instruments that are actively engaged by the input devices, and the instruments that may be coupled to a robotic arm but not actively engaged by the input devices. An instrument may be coupled to a robotic arm but not actively engaged by the input devices when the instrument is assigned to be coupled (e.g., locked in a detachably attachable manner) to the robotic arm. For example, the robotic system may be pre-configured such that certain robotic arms, and thus the attached instruments, are coupled by certain user input devices. An instrument may only be actively engaged by an input device when actions performed at the input devices actually cause movement or manipulation of the robotic arm and/or the attached instrument.

For example, a surgeon may perform a procedure on a patient by thinking in terms of how to manipulate an instrument shown in the display device using either a left HID operated by a left hand of the surgeon or a right HID operated by a right hand of the surgeon. That is, the medical provider's hands may essentially drive and manipulate the instruments by controlling the HIDs. Therefore, the handedness of the console is particularly significant to the medical provider during performance of the procedure.

However, menus that are displayed to the medical provider during the procedure may not display the handedness of the system in an easy to understand format that is also spatially conservative (i.e., minimizes display size at the display device 242). In addition, these menus may not indicate a difference between the instruments that are actively engaged by the HIDs, and the instruments that may be coupled to a robotic arm but not actively engaged by the HIDs. An instrument may be actively engaged by a HID when the instrument is coupled (e.g., locked in a detachably attachable manner) to a robotic arm, and when actions performed at the HIDs (or at any of the actuators/buttons on the HIDs) causes movement or manipulation of the robotic arm and/or the instrument. In contrast, an instrument may not be actively engaged by a HID when the instrument is coupled to a robotic arm, but not currently controllable by a HID.

Failing to indicate the handedness of the robotic system may be problematic for various reasons. For example, failing to indicate the handedness of the robotic system to the medical provider may cause confusion to the medical provider and may result in delays during performance of the procedure. The medical provider may need extra time before the procedure to either manually identify the instrument that is controlled by each HID, or manually test the HIDs to identify the instrument that moves with the movement of the HIDs, which may be shown in the display device. This may especially be the case in surgical robotic systems in which a positioning of the robotic arms may not be indicated in a linear and sequential fashion. The robotic arms may not be indicated in a linear and sequential fashion when, for example, the robotic arms translate around the surgical table such that an order of the robotic arms from one side (i.e., left) to another side (i.e., right) is not always sequential.

Failing to indicate the handedness of the robotic system may also cause unnecessary processing and communications resources to be consumed during the set up process and while performing a procedure. For example, when the user of the console is not aware of the HID control of the instruments, the user may have to reconfigure the setup of the console to manually specify the instrument to be controlled by the specific HIDs. If the user had been aware of the hand assignments for the instruments, the processing and communication load involved in reconfiguration the HID setup could have been avoided.

### C. Introduction to an Interactive Menu Displayed at the Console

The present disclosure provides a technical solution to the foregoing technical problem related to robotic systems and platforms by providing an interactive menu to be displayed at the display device. The interactive menu may indicate the handedness of the system (i.e., indicates the instruments actively engaged with and controlled by a respective HID). In some embodiments, the interactive menu may include an active zone element including indicators related to one or more instruments actively engaged by the HIDs. The active zone element may also include data related to a camera device coupled to one of the robotic arms. The interactive menu may be overlaid on the image displayed at the display device. The active zone element may also include segments or indicators to the right and the left of the scope data to visually represent which instrument is active in each of the medical provider's hands. In this way, the active zone element may combine the indications of the two active instruments and the camera together into a singular unified cluster, which may be positioned on display in a spatially conservative manner.

In some embodiments, the interactive menu may also include an inactive zone element indicating data regarding an inactive instrument that may be coupled to a robotic arm and assigned to be controlled by a HID, but may not be actively engaged (e.g., controlled) by the HID because the HID may, at that moment, be responsible for controlling another active instrument. The inactive zone element may be decoupled from the active zone element, but proximally located with whichever hand the inactive instrument or inactive robotic arm is assigned. The decoupling and positioning of the inactive zone element may emphasize to the medical provider that the inactive instrument is in an inactive state (e.g., may not be controlled by a HID). This may also indicate to the medical provider the HID (and thus the corresponding medical provider hand) to which the inactive instrument is assigned.

### D. Interactive Menu

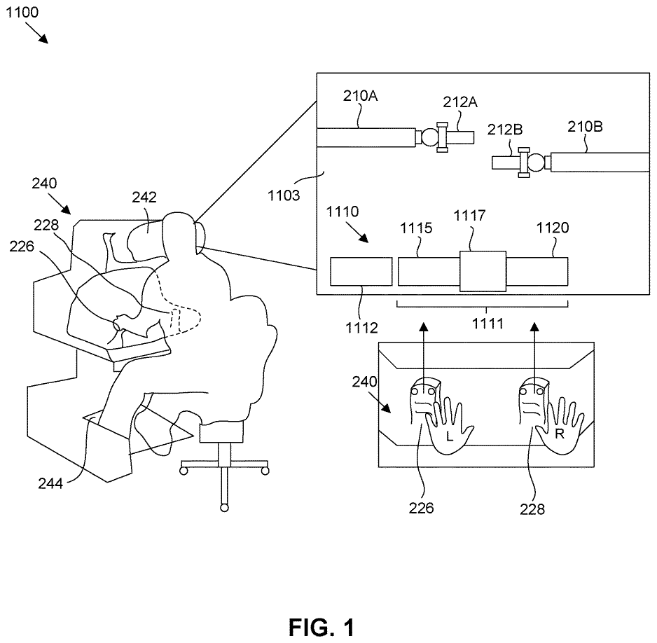

FIG. 1 is a diagram 1100 illustrating the console 240 displaying an image 1103, over which an interactive menu 1110 is presented according to various embodiments of the disclosure. FIG. 1 also illustrates that the interactive menu 1110 presents a hand association between the HIDs 226, 228 and instruments 212A-212B coupled to robotic arms 210A-210B (i.e., shows the handedness of the HIDs 226, 228 to various instruments 212A-212B). The console 240 in FIG. 1 is shown to include the display device 242, a left HID 226 (also referred to herein as a “first HID 226), a right HID 228 (also referred to herein as a “second HID 228”), and a foot pedal assembly 244. However, it should be appreciated that the console 240 may include other components that are not otherwise shown or described in FIG. 1.

In the example shown in FIG. 1, a medical provider may be seated at the console 240 with his or her head immersed into the display device 242. A left hand of the medical provider may operate the left HID 226, and a right hand of the medical provider may operate the right HID 228. Each of the HIDs 226, 228 may be assigned to control multiple robotic arms 210A-210B, but may only be permitted to actively control one robotic arm 210A-210B at a time, and thus one instrument 212A-212B coupled to the robotic arm 210A-210B at a time. The robotic arms 210A-210B and instruments 212A-212B that are actively engaged with and controlled by the HIDs 226, 228 may occasionally be referred to herein as “active robotic arms 210” and “active instruments 212.”

The display device 242 may display the image 1103. The image 1103 may be an image of a patient anatomy captured by a camera, such as, for example, an endoscopic camera or a laparoscopic camera. In one case, the camera may be coupled to one of the robotic arms 210A-210B and capturing the image 1103 as a live-stream while the medical provider uses the surgical robotic system to perform a procedure on the patient. In another case, another camera may have captured the image 1103 prior to performing a procedure on the patient using the surgical robotic system.

The image 1103 may not only display a portion of the patient anatomy, but may, in some cases, also display one or more active instruments 212A-212B that are actively engaged by the HIDs 226 and 228. In the example shown in FIG. 1, a first instrument 212A and a second instrument 212B are displayed in the image 1103, in which the two instruments 212A-212B may be different or may be the same. In the example shown in FIG. 1, a first robotic arm 210A is shown as coupled to the first instrument 212A, while a second robotic arm 210B is shown as coupled to the second instrument 212B. FIG. 1 shows the image 1103 as depicting a portion of the robotic arms 210A-210B for illustrative purposes only, and it should be appreciated that the robotic arms 210A-210B may sometimes not be displayed in an image 1103. In these cases, only the end effectors of the instruments 212A-212B may be displayed in the image 1103.

The interactive menu 1110 may be overlaid on top of the image 1103. As shown in FIG. 1, the interactive menu 1110 may be positioned at the bottom edge of the image 1103. A size of the interactive menu 1110 may be a small percentage of a size of the image 1103. However, the size of the interactive menu 1110 may be adjustable via, for example, a user interaction at the console 240, to either increase or decrease the size of the interactive menu 1110. In some embodiments, the size of interactive menu 1110 may be less than or equal to a maximum proportion or percentage of the size of the image 1103, so as to ensure that the interactive menu 1110 does not distract the medical provider from focusing on the procedure control using the instruments 212A-212B displayed in the image 1103.

The interactive menu 1110 may include an active zone element 1111 and an inactive zone element 1112. The active zone element 1111 may indicate information regarding active instruments 212A-212B actively controlled by the HIDs 226, 228. The inactive zone element 1112 may indicate information regarding the inactive instruments 212 that may be coupled a robotic arm 210 but not actively controlled by the HIDs 226, 228.

As shown in FIG. 1, the active zone element 1111 may include a left segment 1115 (also sometimes referred to herein as a “first segment 1115”), a center segment 1117, and a right segment 1120 (also sometimes referred to herein as a “second segment 1120”). The left segment 1115 may include data describing an active instrument 212A controlled by the left HID 226. The left HID 226 may be assigned to and actively controlling a first robotic arm 210A coupled to the first active instrument 212A. The right segment 1120 may include data describing an active instrument 212B controlled by the right HID 228. The right HID 228 may be assigned to and actively controlling a second robotic arm 210A coupled to the second active instrument 212B.

As shown in FIG. 1, the interactive menu 1110 may indicate the hand associations between the HID 226, 228 and the segments 1115 and 1120. Specifically, the left segment 1115 indicates that the instrument 212A is controlled by the left HID 226 and operated by the user's left hand. Similarly, the right segment 1120 indicates that the instrument 212B is controlled by the right HID 228 and operated by the user's right hand. While FIG. 1 shows the instrument 212A being positioned on the left side of the image 1103 and the instrument 212B being positioned on the right side of the image 1103, the positioning of the instruments 212A-212B themselves within the image 1103 may not be relevant to the interactive menu 1110. For example, the positioning of the data within the active zone element 1111 may remain the same, even when the instrument 212A is positioned on the right side of the image 1103 and the instrument 212B is positioned on the left side of the image 1103. In this case, the interactive menu 1110 may still indicate that the HID 226 may be used to control instrument 212A and the HID 228 may be used to control instrument 212B. Hereinafter, the term “instrument 212A-212B” may occasionally be referred to as “instrument 212,” and the term “robotic arm 210A-210B” may occasionally be referred to as “robotic arm 210.”

The inactive zone element 1112 may indicate data associated with the inactive instrument 212. As shown in FIG. 1, the inactive zone element 1112 is positioned outside the active zone element 1111, but proximate to the left segment 1115, indicating that the inactive instrument 212 is assigned to the left HID 226. A visual factor of the inactive zone element 1112 may also indicate that the inactive instrument 212 is not currently being controlled by the left HID 226. For example, the visual factor may be a brightness level or a color of the inactive zone element 1112 or at least a portion of a border of the inactive zone element 1112. A brightness level of the inactive zone element 1112 may be less bright than a brightness level of the active zone element 1111. A color of the inactive zone element 1112 may blend into the background of the image 1103 to dull out the information in the inactive zone element, while a color of the active zone element 1111 may contrast with the background of the image 1103, to emphasize the information in the active zone element 1111. In this way, the interactive menu 1110 includes two primary components, the active zone element 1111 and the inactive zone element 1112, which together illustrate the handedness of the system to the user in a compact, easy to digest format.

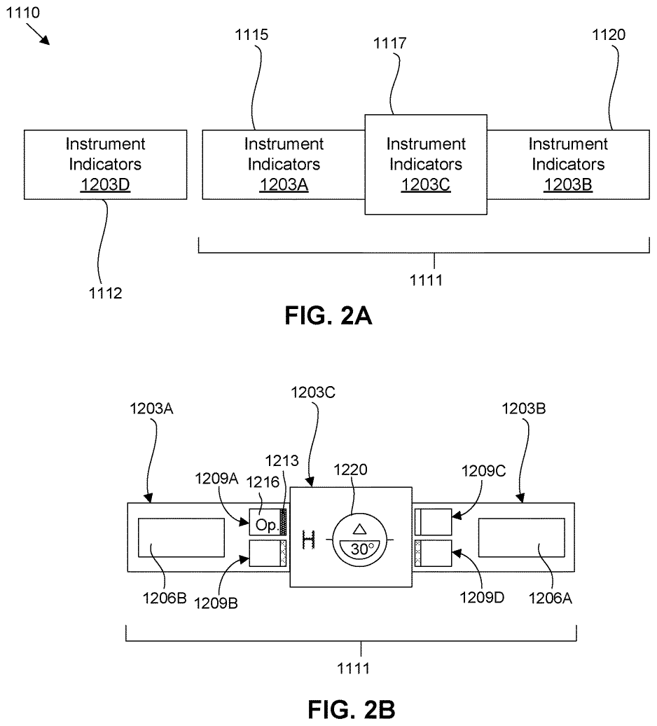

FIGS. 2A and 2B are diagrams illustrating the interactive menu 1110 according to various embodiments of the disclosure. Referring specifically to FIG. 2A, the interactive menu 1110 may include the active zone element 1111 and the inactive zone element 1112. The active zone element 1111 may include the left segment 1115, the center segment 1117, and the right segment 1120.

A visual factor of the active zone element 1111 may indicate that the instruments 212A-212B described in the active zone element 1111 are actively engaged with the HIDs 226, 228. For example, the visual factors of the active zone element 1111 may be displayed more predominantly than the visual factors of the inactive zone element 1112, to emphasize that instruments 212 indicated in the active zone element 1111 are active while the instrument 212 indicated in the inactive zone element 1112 is not active. To this end, the active zone element 1111 may be displayed with, for example, a greater brightness or a different color than the inactive zone element 1112. For example, the colors used to display the different components, the border of the components, and text in the active zone element 1111 and/or the background colors behind the text and icons in the active zone element 1111 may have a higher brightness level or higher contrast than those in the inactive zone element 1112. The size/thickness of the text in the active zone element 1111 may be larger than the size/thickness of the text in the inactive zone element 1112. In this way, the inactive zone element 1112 is dimmed or dulled out relative to the active zone element 1111, such that the inactive zone element 1112 does not distract the user from focusing on the image 1103 or the data indicated in the active zone element 1111.

As shown in FIG. 2A, the inactive zone element 1112 is positioned proximate to the left segment 1115 of the active zone element 1111. This may signal to the user that the instrument 212 described in the inactive zone element 1112 may be controlled by a robotic arm 210 assigned to be operated by the left HID 226. As further described herein, the user may actuate a user input device at the console 240 to switch the control of the left HID 226 from the instrument 212 indicated in the left segment 1115 to the instrument 212 indicated in the inactive zone element 1112.

Each of the segments 1115, 1117, and 1120 of the active zone element 1111 may include instrument indicators 1203A, 1203B, and 1203C, respectively. The instrument indicators 1203A of the left segment 1115 may indicate data regarding a first instrument 212A coupled to a first robotic arm 210A, which may be controlled by a first HID 226. When the left segment 1115 is displayed on a left side of the center segment 1117, the user may determine that the first instrument 212A indicated in the left segment 1115 may be controlled by the first HID 226, since the first HID 226 may be operated by the user's left hand. The instrument indicators 1203B of the right segment 1120 may indicate data regarding a second instrument 212B coupled to a second robotic arm 210B, which may be controlled by a second HID 228. When the right segment 1120 is displayed on a right side of the center segment 1117, the user may determine that the second instrument 212B indicated in the right segment 1120 may be controlled by the second HID 228, since the second HID 228 may be operated by the user's right hand. The instrument indicators 1203C of the center segment 1117 may indicate scope data describing a camera coupled to a third robotic arm 210. The instrument indicators 1203D of the inactive zone element 1112 may indicate data regarding a third instrument 212 coupled to a fourth robotic arm 210, which may be an inactive robotic arm 210 (i.e., not actively controlled by either of the HIDs 226, 228, and may be, for example, resting at the side of the system and/or locked into place to prevent accidental manual movement of the inactive robotic arm 210).

Referring specifically now to FIG. 2B, each of the instrument indicators 1203A-1203C may include text 1206A-B and/or icons 1209A-D, 1220 that indicate data regarding an active instrument 212A-212B coupled to an active robotic arm 210A-210B. For example, the instrument indicator 1203A describing the first instrument 212A coupled to the first robotic arm 210A may include, for example, an identifier of the first robotic arm 210A, an identification of the first instrument 212A, icons 1209A-D representing one or more operations that may be performed using the first instrument 212A, and/or any other information describing the first instrument 212A and/or the first robotic arm 210A. The identifier of the first robotic arm 210A may be represented as text 1206 including one or more alphanumeric values identifying the first robotic arm 210A. The identification of the first instrument 212A may be represented as text 1206 reciting a name of the first instrument 212A. This identification may be easily and immediately understood by the user as the first instrument 212A, particularly when the first instrument 212A is also displayed in the image 1103.

The icons 1209A-D may represent information describing one or more operations to be performed or currently performed using the instruments 212A-B. Each of the icons 1209A-D may include a dynamic border 1213 and text 1216 (although only icon 1209A in FIG. 2B is shown with the reference numbers 1213 and 1216, it should be appreciated that icons 1209B, 1209C, and 1209D also include a dynamic border 1213 and text 1216. The text 1216 may describe the type of operation indicated by a respective icon 1209A-D. Each dynamic border 1213 may display certain visual features or attributes based on an operation to be performed or currently being performed by a respective instrument 212A-B. As shown in FIG. 2B, the dynamic borders 1213 at the icons 1209A-B may be positioned on one edge of each of the icons 1209A-B, proximate to or abutting the center segment 1117. Similarly, the dynamic borders 1213 at icons 1209C-D may also be positioned at an edge of the icons 1209C-D, proximate to or abutting the center segment 1117. However, it should be appreciated that other edges of the icons 1209A-D or multiple edges of the icons 1209A-D may also include similar dynamic borders 1213. The icons 1209A-D may also be positioned at an edge of the instrument indicators 1203A-D, proximate to or abutting the center segment 1117. In this way, the dynamic borders 1213 and the icons 1209A-D are positioned in the center of the active zone element 1111, such that the information depicted by the icons 1209A-D are displayed in a central but non-distracting manner at the display device 242.

For example, each of the dynamic borders 1213 may be set to a particular color, shading, transparency, pattern, or other type of visual attribute to indicate a type of operation represented by the icon 1209A-D, a step of performing the operation (e.g., setting up to perform, actually performing, not performing, etc.), or other action related to the operation. The attributes of the dynamic borders 1213 may change based on a status of the operation (e.g., whether the operation is currently being performed using the instrument 212A-B or not being performed). As shown in FIG. 2B, each of the dynamic borders 1213 may be different for different types of operations, as a way of indicating a type of operation to the user. In some cases, an instrument 212A-D being described by an instrument indicator 1203B may not be capable of performing more than one operation. In such a case, an icon 1209C may be left empty.

Each of the icons 1209A-D may include text 1216 of a pre-defined color and/or a background color of the icon 1209A-D may be of the pre-defined color, either of which may indicate a status of the operation. For example, the background color may change to a first color when a particular operation is about to be activated or performed (e.g., when a foot of the user hovers over an input device at the foot pedal assembly 244), the background color may flash brightly when the location of the first instrument 212A is being positioned to perform the operation, the background color may change to a second color when the operation is actually being performed, etc. The icons 1209A-D may also include images representing the instrument 212A or the operations, in which the images may be provided by a camera or may be rendered images.

As an illustrative example, the medical provider may activate an operation indicated by an icon 1209A via an input device (e.g., button or pedal) at the foot pedal assembly 244. For example, the foot pedal assembly 244 may include a first input device, which, when actuated (e.g., pressed), may activate a cutting operation using the first instrument 212A identified in the instrument indicator 1203A. Similarly, the foot pedal assembly 244 may include a second input device, which, when actuated (e.g., pressed), may activate the coagulation operation using the first instrument 212A identified in the instrument indicator 1203A. In this case, the icon 1209A may be associated with the cutting operating, and the icon 1209B may be associated with the coagulation operation. When the input device is released, for example, the first instrument 212A may terminate performing the respective operation. The background color and/or an attribute of the dynamic border 1213 of the icon 1209A representing the operation may change relative to an actuation of the foot pedal assembly 244 and/or a status of the operation being performed on the patient at the location at which the first instrument 212A is positioned. For example, the dynamic border 1213 of icon 1209A and/or the background color of the icon 1209A may change to a first color when the medical provider hovers over the input device at the foot pedal assembly 244 used to activate the operation, may change again when the medical provider first actuates the input device used to perform the operation, and may remain flashing while the medical provider keeps actuating the input device to continue performing the operation for a period of time. In this way, the icon 1209A-D indicating the operation may include several different ways of signaling information to the user, in a compact format, which may be easily and clearly understood by the user as he or she is performing the operation and viewing the interactive menu 1110.

The instrument indicator 1203B describing the second instrument 212B coupled to the second robotic arm 210B may similarly include, for example, an identifier of the second robotic arm 210B, an identification of the second instrument 212B, an indication of one or more operations that may be performed using the second instrument 212B, and/or any other information describing the second instrument 212B or the second robotic arm 210B. To this end, the instrument indicators 1203B may also include text 1206 to describe the second instrument 212B coupled to the second robotic arm 210B and icons 1209C-D to indicate and describe a state of operations that may be performed by the second instrument 212.

The instrument indicator 1203C describing a third instrument 212, such as, for example, a camera, may also include an identifier of the third robotic arm 210, an identification of the third instrument 212, an indication of one or more operations that may be performed using the third instrument 212, and/or any other information describing the third instrument 212 or the third robotic arm 210, which may be in the form of text 1206. The instrument indicator 1203C may also include an icon 1220 describing settings and a state of a third instrument 212 coupled to the robotic arm 210. In the example shown in FIG. 2B, the third instrument 212 may be a camera or a scope device.

For example, when the HIDs 226, 228 are switched to a camera controlling mode, the HIDs 226, 228 may be locked together in a single plane and moved together to control movement of the third robotic arm 210, and thus control movement of the camera. In this case, the instrument indicator 1203C may include scope data describing the camera used by the robotic system. The scope data may include, for example, an identifier of the third robotic arm 210 and data describing the camera. The scope data may include the icon 1220 representing a camera. For example, the icon 1220 may be embodied as a circle with two wings, in which the two wings may represent an orientation of the camera with respect to a horizontal horizon line (e.g., as indicated by the “H” in the icon 1213). The icon 1220 may rotate depending on a position of the camera in relation to the horizontal horizon line. The icon 1220 may also indicate a type of bezel on a tip of the camera, for example, by indicating an angle of incline of the bezel located at the tip of the endoscopic or laparoscopic device (e.g., as shown by the text 30° in the icon 1213). The icon 1220 may also include an arrow indicating a direction of the camera scoping the patient during the procedure.

The instrument indicator 1203D included in the inactive zone element 1112 describes an inactive instrument 212 coupled to a fourth robotic arm 210, not actively engaged with or controlled by a HID 226, 228. The instrument indicators 1203D may include, for example, an identifier of the fourth robotic arm 210, an identification of the fourth instrument 212, and/or any other information describing the fourth instrument 212 or the fourth robotic arm 210. To this end, the instrument indicators 1203D may also include text 1206 describing the fourth instrument 212 coupled to the fourth robotic arm 210.

In an embodiment, the inactive zone element 1112 and the segments 1115, 1117, and 1120 may each include dynamic borders, which may be highlighted in various colors and/or shading to indicate whether the respective instrument 212 is engaged with or able to be controlled by a HID 226, 228. The dynamic border may be positioned around one or more edges of the inactive zone element 1112 or the segments 1115, 1117, 1120. For example, the dynamic border of the left segment 1115 may be blue when the left HID 226 is coupled to and engaged with the first instrument 212A such that the left HID 226 may be used to control the first instrument 212A. In contrast, the dynamic border of the left segment 1115 may be black or grey when the left HID 226 is disengaged and may not be used to control the first instrument 212A.

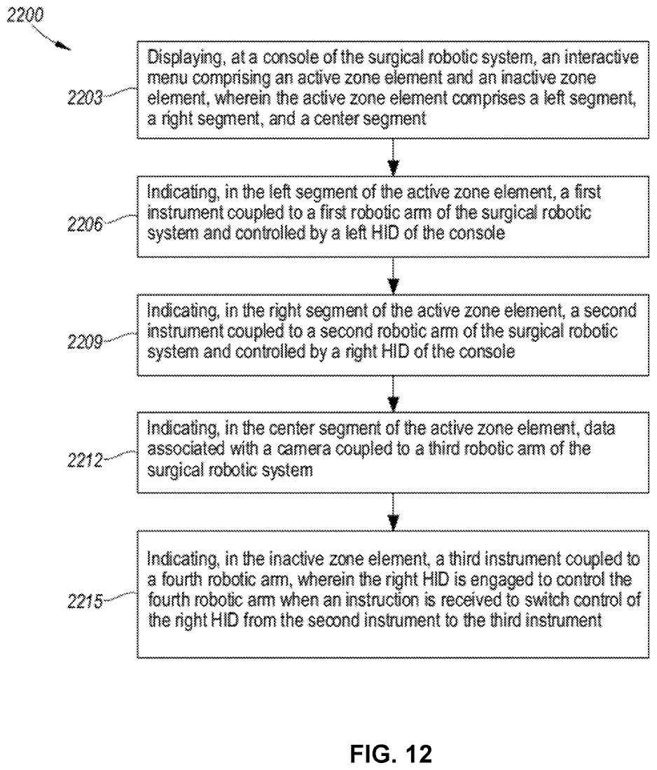

While the interactive menu 1110 shown in FIG. 2 only includes one inactive zone element 1112 decoupled from the active zone element 1111, it should be appreciated that the interactive menu 1110 may include another inactive zone element 1112, for example, positioned proximate to and separate from the right segment 1120. While FIG. 12 shows various components and text included in the interactive menu 1110, it should be appreciated that the interactive menu 1110 may include several other types of data, in the form of icons, colors, text, and/or other indicators, which may be used to describe an instrument 212, robotic arm 210, and/or an operation.

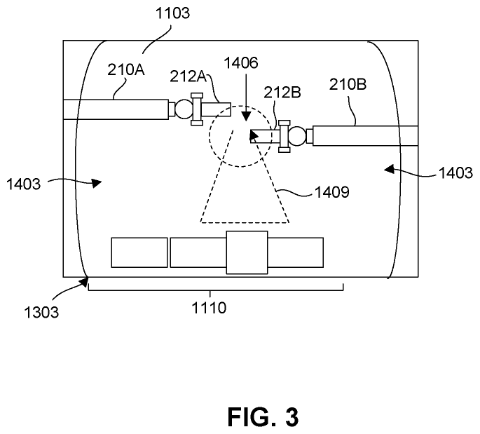

FIG. 3 illustrates an example image 1103 and interactive menu 1110 according to various embodiments of the disclosure. The image 1103 and interactive menu 1110 may be displayed at the display device 242 of the console 240.

The interactive menu 1110 may be overlaid on the image 1103, as shown in FIG. 3. Alternatively, the interactive menu 1110 may be displayed as a separate window decoupled from the image 1103, such that the user may actuate an input device at the console 240 to switch between the image 1103 and the interactive menu 1110.

As shown in FIG. 3, the interactive menu 1110 may be positioned at the bottom edge 1303 of the image 1103. The interactive menu 1110 may otherwise be positioned proximate to any of the other edges of the image 1103, so long as the interactive menu 1110 is overlaid on the image 1103 in a non-distracting manner.

A size of the interactive menu 1110 may be a percentage of a size of the image 1103. However, the size of the interactive menu 1110 may be adjustable via a user input at the console 240, to either increase or decrease the size of the interactive menu 1110. In some embodiments, the size of interactive menu 1110 may be less than or equal to a preset proportion or percentage of the size of the image 1103, so as to ensure that the interactive menu 1110 does not distract the user from focusing on the tasks performed by the instruments 212 displayed in the image 1103.

FIG. 3 also illustrates an example of a medical provider's eye movement while viewing an image 1103 and interactive menu 1110 according to various embodiments of the disclosure. While the display device 242 may display the entire image 1103, the user may primarily, and most easily, view the area 1403 when a head of the user is immersed within the display device 242. In this way, the area 1403 may represent an approximation of the user's general field of vision when the user's head is immersed within the display device 242. Meanwhile, the area within circle 1406 may represent an approximation of the user's focus area or central point of vision when the user's head is immersed within the display device 242. For example, the area within circle 1406 may present an anatomy of the patient in which a procedure is being performed or is to-be performed by active instruments 212A and 212B.

As shown in FIG. 3, the components of the interactive menu 1110 or the information indicated in the interactive menu 1110 is not distributed throughout the image 1103 or screen, but is instead compactly presented at one location. In this way, the user may view, read, and digest the information in the interactive menu 1110 quickly, without straining the user's eyes, and without distracting the user from the area within circle 1406 or area 1403.

An example of a medical provider's eye movement while performing a procedure on a patient and viewing an image 1103 and interactive menu 1110 is presented in FIG. 3 as arrow 1409. The positioning of the interactive menu 1110 at the bottom edge 1303 of the image 1103 or screen enables the interactive menu 1110 to be positioned within area 1403, or positioned within the medical provider's general field of vision when immersed in the display device 242. In addition, the position of the interactive menu 1110 may be proximate to the circle 1406, such that viewing and interacting with the interactive menu 1110 may not necessarily distract the medical provider from the primary area of the procedure.

### E. Interactive Menu Updates Based on User Input

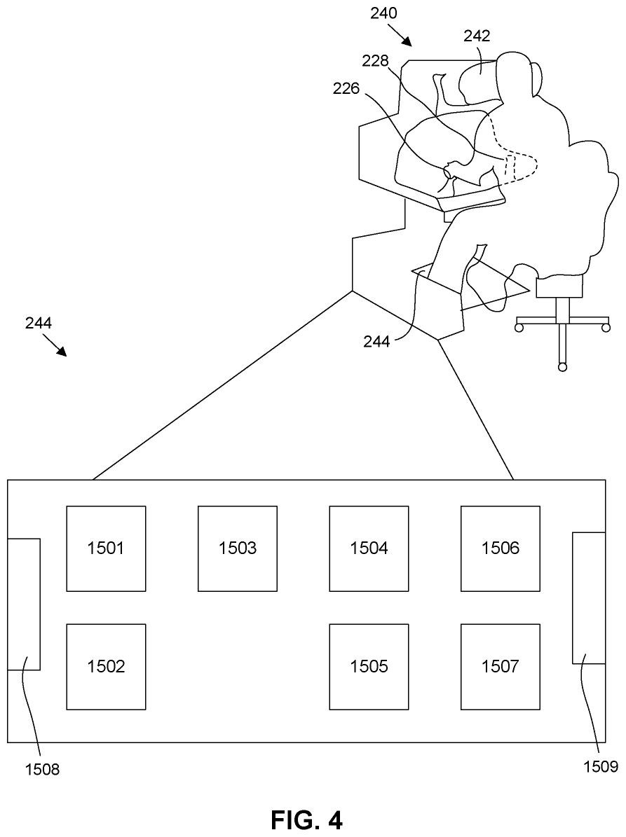

FIGS. 4-8C below describe one or more user input devices, which when actuated or released, may cause certain updates to be displayed at the interactive menu 1110. The example user input devices described below with regard to FIGS. 4-8C are pedals on a foot pedal assembly 244. However, it should be appreciated that the interactive menu 1110 updates shown in FIGS. 4-8C may be presented in response to user interactions with any other type of user input device (e.g., touchscreen enabled displays, switches/buttons on the HIDs 226, 228, switches/buttons on a hand rest of the console 240, etc.). Therefore, the foot pedal assembly 244 is used in the examples of FIGS. 4-8C for illustrative purposes only. However, the user input devices that may cause updates to an interactive menu 1110 should not be limited to pedals in the foot pedal assembly 244.

Nevertheless, FIG. 4 is a diagram illustrating a foot pedal assembly 244 that may be used to operate various modes of the instruments 212 and cameras controlled by the robotic systems according to various embodiments of the disclosure. As shown in FIG. 4, the foot pedal assembly 244 includes multiple input devices 1501-1509, which may be buttons, pedals, switches, touch sensitive surfaces, etc. Each of the input devices 1501-1509, when actuated, may implement a mode, perform an operation, or adjust a display at the display device 242. While only nine input devices 1501-1509 are shown in FIG. 4, it should be appreciated that the foot pedal assembly 244 may include any number of input devices 1501-1509.

In the embodiment shown in FIG. 4, the foot pedal assembly 244 may be part of a console 240 of a surgical robotic system. In this case, the foot pedal assembly 244 may include, for example, a clutch input device 1501, a camera input device 1502, a menu input device 1503, a top-left input device 1504, a bottom-left input device 1505, a top-right input device 1506, a bottom-right input device 1507, a left instrument switch input device 1508, and a right instrument switch input device 1509.

The clutch input device 1501, when actuated, may disengage a HID 226, 228 from a respective robotic arm 210. The camera input device 1502, when actuated, may lock both HIDs 226, 228 together such that both HIDs 226, 228 may only be moved together in the same motion, which may be used to control movement of a camera coupled to a robotic arm 210.

The menu input device 1503, when actuated, may instruct the display device 242 to display the interactive menu 1110 or to remove the interactive menu 1110 from display. This may be in the form of overlaying the interactive menu 1110 over the image 1103 or presenting the interactive menu 1110 as a standalone display, separate from the image 1103. In the latter case, actuation of the menu input device 1503 may alternate between displaying the image 1103 and displaying the interactive menu 1110.

The top-left input device 1504 and the bottom-left input device 1505 may each correspond to different operations that may be performed by a first active instrument 212A controlled by the left HID 226. Referring back to FIG. 2, the interactive menu 1110 may include icons 1209A-D each representing operations that may be performed by a respective instrument 212. Each operation for the first active instrument 212A controlled by the left HID 226 may be initiated and/or activated using either the top-left input device 1504 or the bottom-left input device 1505.

Similarly, the top-right input device 1506 and the bottom-right input device 1507 may each correspond to different operations that may be performed by a second active instrument 212B controlled by the right HID 228. Each operation for the second active instrument 212B controlled by the right HID 228 may be initiated and/or activated using either the top-right input device 1506 or the bottom-right input device 1507.

The left instrument switch input device 1508, when actuated, may instruct the console 240 to move the instrument indicators 1203D describing the inactive instrument 212 (configured to be controlled by the left HID 226) from the inactive zone element 1112 to the left segment 1115, and move instrument indicators 1203A describing the instrument 212A from the left segment 1115 to the inactive zone element 1112. In this case, the inactive zone element 1112 may be positioned proximate to and to the left side of the left segment 1115. The right instrument switch input device 1509, when actuated, may instruct the console 240 to move instrument indicators 1203A-1203D describing an inactive instrument 212 (configured to be controlled by the right HID 228) from the inactive zone element 1112 to the right segment 1120, and move the instrument indicators 1203B describing the instrument 212B from the right segment 1120 to the inactive zone element 1112. In this latter case, the inactive zone element 1112 may be positioned proximate to and to the right side of the right segment 1120.

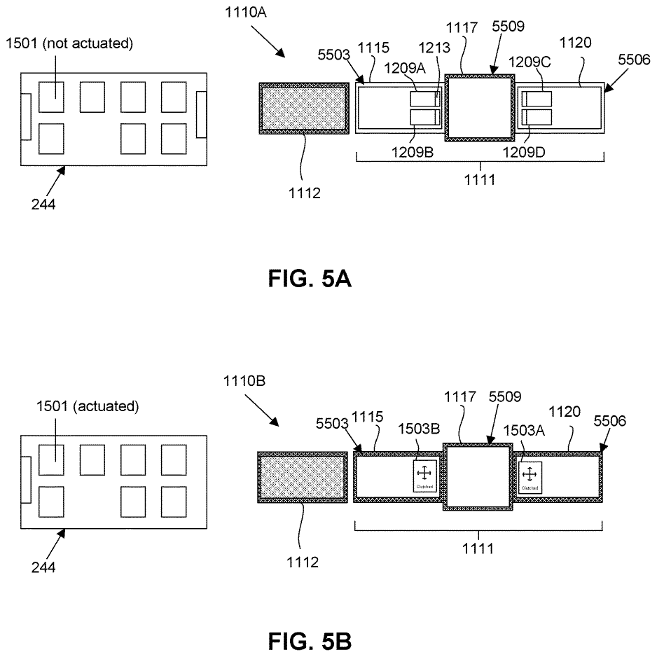

FIGS. 5A and 5B illustrate various embodiments of the interactive menu 1110 dynamically changing in response to actuation of a user input device at the foot pedal assembly 244 according to various embodiments of the disclosure. The actuated user input device in FIGS. 5A and 5B is the clutch input device 1501. Specifically, FIG. 5A shows an embodiment of the interactive menu 1110A when the clutch input device 1501 of the foot pedal assembly 244 is not actuated, and FIG. 5B shows an embodiment of the interactive menu 1110B when the clutch input device 1501 of the foot pedal assembly 244 is actuated.

The segments 1115, 1117, and 1120 in the interactive menus 1110A-B shown in FIGS. 5A-B each include dynamic borders 5503, 5506, and 5509, respectively. The dynamic border 5503 at least partially surrounds the left segment 1115 (or the edges of the left segment 1115 that do not abut the center segment 1117). The dynamic border 5506 at least partially surrounds the right segment 1120 (or the edges of the right segment 1120 that do not abut the center segment 1117). The dynamic border 5509 at least partially surrounds the center segment 1117 (or the top and bottom edges of the center segment 1117 that do not abut the left segment 1115 and the right segment 1120).

FIG. 5A shows an example of the interactive menu 1110A when the clutch input device 1501 is not actuated. As shown in FIG. 5A, a dynamic border 5503 of the left segment 1115 and the dynamic border 5506 of the right segment 1120 remain activated and highlighted, indicating that the instruments 212A-B indicated in the left segment 1115 and the right segment 1120 are engaged to be controlled by the HIDs 226, 228. For example, the dynamic borders 5503 and 5506 are indicated in FIG. 5A as being activated and highlighted by depicting the borders 5503 and 5506 as white, without any shading. Meanwhile, the dynamic border 5509 of the center segment 1117 is deactivated, indicating that the instrument 212 described in the center segment 1117 is not engaged to be controlled by the HIDs 226, 228. For example, the dynamic border 5509 is indicated as being deactivated by depicting the border 5509 as grey and shaded. In addition, the icons 1209A-D, and the dynamic borders 1213 of the icons 1209A-D, in the respective segments 1115 and 1120 are also shown as being activated and highlighted in the interactive menu 1110A, indicating that the operations represented by the icons 1209A-D may be performed using the instruments 212A-B. Again, the icons 1209A-D and the dynamic borders 1213 of the icons 1209A-D are indicated in FIG. 5A as being activated and highlighted by depicting white icons 1209A-D and dynamic borders 1213, without any shading.

FIG. 5B shows an example of the interactive menu 1110B when the clutch input device 1501 is actuated. As shown in FIG. 5B, the dynamic border 5503 of the left segment 1115 and the dynamic border 5506 of the right segment 1120 change to be deactivated, indicating that the instruments 212A-B indicated in the left segment 1115 and the right segment 1120 are not engaged to be controlled by the HIDs 226, 228. The dynamic border 5509 of the center segment 1117 remains deactivated, indicating again that the instrument 212 described in the center segment 1117 is again not engaged to be controlled by the HIDs 226, 228. The dynamic borders 5503, 5509, and 5509 are indicated in FIG. 5B as being deactivated by depicting the borders 5503, 5506, and 5509 as grey and shaded (i.e., shading the borders 5503, 5506, and 5509 in grey, or another darker color).

In addition, the left segment 1115 and the right segment 1120 both include a clutched icon 1503 instead of the icons 1209A-D. In other words, the area of each of the instrument indicators 1203A-B, which previously included two icons 1209A-D, may instead include a single clutched icon 1503. The size of the clutched icon 1503 may be the same as the size of two icons 1209A-B or 1209C-D. As shown in FIG. 5B, the clutched icon 1503 may include a rendered icon with opposing arrows and/or text stating the term “clutched.” In this way, it may be easily and immediately determined that the instruments 212A-B described in the left segment 1115 and right segment 1120 are not only disengaged from the HIDs 226, 228, but also clutched at bedside.

While the activation and deactivation of the segments 1115, 1117, and 1120, dynamic borders 1203, 5503, 5506, 5509, and icons 1209A-D are described with respect to FIGS. 5A-5B as being displayed in either white or dark grey, it should be appreciated that the activation and deactivation of the segments 1115, 1117, and 1120, dynamic borders 1203, 5503, 5506, 5509, and icons 1209A-D may be indicated in a variety of other different manners (e.g., using colors, flashing, shading, etc.), which should not be otherwise limited herein.

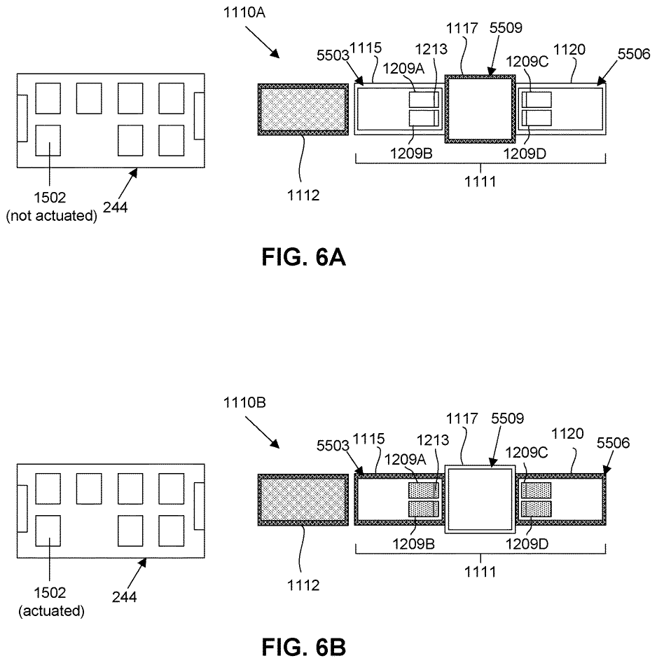

FIGS. 6A and 6B illustrate various embodiments of the interactive menu 1110 dynamically changing in response to actuation of a user input device at the foot pedal assembly 244 according to various embodiments of the disclosure. The actuated user input device in FIGS. 6A and 6B is the camera input device 1502. Specifically, FIG. 6A shows an embodiment of the interactive menu 1110A when the camera input device 1502 of the foot pedal assembly 244 is not actuated, and FIG. 6B shows an embodiment of the interactive menu 1110B when the camera input device 1502 of the foot pedal assembly 244 is actuated.

As shown in the interactive menu 1110A of FIG. 6A, when the camera input device 1502 is not actuated, visual factors of the dynamic border 5503 around at least a portion of the left segment 1115 and the dynamic border 5506 around at least a portion of the right segment 1120 remain activated (e.g., presented in a color or brightness, for example, emphasizing the left segment 1115 and the right segment 1120 to the user). As shown in FIG. 6A, the dynamic border 5503 of the left segment 1115 is activated and remains highlighted in a brighter color (e.g., shown in white and not shaded in grey), and the dynamic border 5506 of the right segment 1120 is similarly activated and remains highlighted, indicating that the instruments 212 identified in the left segment 1115 and the right segment 1120 are actively controlled by HIDs 226, 228. In addition, the respective segments 1115 and 1120 may also display instrument indicators 1203A and 1203B (e.g., text 1206 or icons 1209A-D) indicating operations that may be performed by the active instruments 212. As shown in FIG. 6A, icons 1209A-B are also activated and highlighted (e.g., shown in white and not shaded in grey), such that the data included in the icons 1209A-B are easily visible to the user. Meanwhile, the dynamic border 5509 of the center segment 1117 is deactivated and displayed with a darker color (e.g., shaded in grey), indicating that the instrument 212 (e.g., the camera) is not being actively controlled by the HIDs 226, 228 at the time of displaying the interactive menu 1110A of FIG. 6A.

In contrast, as shown in the interactive menu 1110B of FIG. 6B, when the camera input device 1502 is actuated, visual factors of the dynamic border 5503 and the dynamic border 5506 around at least a portion of the left segment 1115 and the right segment 1120 may be de-activated (e.g., shaded in grey). As shown in FIG. 6B, the dynamic border 5503 of the left segment 1115 and the dynamic border 5506 of the right segment 1120 are both shaded, indicating that the instruments 212 identified in the left segment 1115 and the right segment 1120 are not currently actively controlled by HIDs 226, 228 at the time of displaying the interactive menu 1110B of FIG. 6B. As mentioned above, presenting the dynamic borders 5503, 5506 in this manner may help signal to the user that the left HID 226 and right HID 228 may have been disengaged, and thus may not be operated to control the instruments 212 indicated in the left segment 1115 and right segment 1120, respectively. Meanwhile, the dynamic border 5509 of the center segment 1117 may change to be activated and highlighted (e.g., shown in white), indicating that the instrument 212 (e.g., the camera) is indeed being actively controlled by the HIDs 226, 228 at the time of displaying the interactive menu 1110B of FIG. 6B. Presenting the dynamic border 5509 of the center segment 1117 in this manner may help signal to the user that the left HID 226 and right HID 228 are now engaged with the instrument 212 (e.g., the camera) identified and described in the center segment 1117. While the activation and deactivation of the dynamic borders 5503, 5506, and 5509 are described with respect to FIGS. 6A-6B as being displayed in either white or dark grey, it should be appreciated that the activation and deactivation of the dynamic borders 5503, 5506, and 5509 may be indicated in the dynamic borders 5503, 5506, and 5509 in a variety of other different manners (e.g., using colors, flashing, shading, etc.), which should not be otherwise limited herein.

Similarly, when the camera input device 1502 is actuated, the icons 1209A-D in the left segment 1115 and the right segment 1120 may change to indicate that operations may no longer be performed since the instruments 212A-B indicated in the left segment 1115 and the right segment 1120 have been disengaged from the HIDs 226, 228. As shown in FIG. 6B, the icons 1209A-D may be shaded in a darker color, such as grey, to dull out the data displayed in the icons 1209A-D. Similarly, the dynamic borders 1213 on the icons 1209A-D may also be deactivated by shading the dynamic borders 1213 to a similar dark color, which may or may not be the same shade as the remainder of the icon 1209A-D. that previously presented operations that may be performed by the instruments 212 may be replaced with an indication that the instrument 212 is clutched, and as such, the HIDs 226, 228 are disengaged from the robotic arms 210 coupled to the instruments 212. The indication that the instrument 212 is clutched may be represented using text 1206 and/or an icon 1209, in which the icon 1209 may be an image or a rendering.

While the activation and deactivation of the dynamic borders 5503, 5506, and 5509, 1213 and icons 1209A-D are described with respect to FIGS. 6A-6B as being displayed in either white or dark grey, it should be appreciated that the activation and deactivation of the dynamic borders 5503, 5506, and 5509, 1213 and icons 1209A-D may be indicated in a variety of other different manners (e.g., using colors, flashing, shading, etc.), which should not be otherwise limited herein.

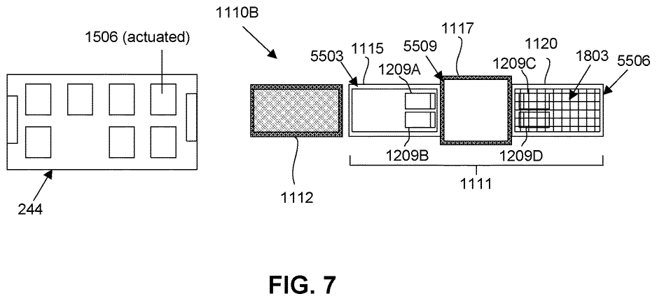

FIG. 7 illustrates an embodiment of the interactive menu 1110 when the top-right input device 1506 of the foot pedal assembly 244 is actuated according to various embodiments of the disclosure. As shown in FIG. 7, the dynamic border 5509 around the center segment 1117 is deactivated (shaded in grey), which the dynamic border 5503 around the left segment 1115, and the dynamic border 5506 around the right segment 1120 are activated (depicted in white). The shading of the dynamic borders 5503, 5506, and 5509 indicate the instruments 212 that are actively engaged to be controlled by the HIDs 226, 228. That is, the instruments 212 indicated in the left segment 1115 and the right segment 1120, with activated dynamic borders 5503, 5506, are actively controlled by the HIDs 226, 228. Meanwhile, the instrument 212 indicated in the center segment 1117, with the deactivated dynamic border 5509, are not actively controlled by the HIDs 226, 228.

As mentioned above, the right segment 1120 may include one or more icons 1209C-D related to one or more operations performed by the instrument 212 indicated in the right segment 1120. An icon 1209C-D displayed in the right segment 1120 may correspond to the top-right input device 1506, such that actuation of the top-right input device 1506 corresponds to the performing of the operation indicated by the icon 1209C-D. When the top-right input device 1506 is actuated, an operation indicated in by an icon 1209C-D in the right segment 1120 may be performed using the instrument 212 indicated in the right segment 1120. When the operation is being performed (i.e., as the top-right input device 1506 is being actuated), the interactive menu 1110 may drastically change the visual factors of the right segment 1120. For example, colors and shading of the right segment 1120, and/or the interactive menu 1110 may even begin flashing to clearly indicate to the user that an operation is being performed using the instrument 212. In some cases, a sound alert may also be output by the console 240 when the top-right input device 1506 is actuated. For example, such alerts may be significantly helpful to a medical provider to ensure that the operations are performed at the most accurate and opportune moment, to prevent injury to the patient and other severe issues. In this way, the interactive menu 1110 may provide alerts to the medical provider in response to operations being performed using a particular active instrument 212.

In an embodiment, the visual factors of the dynamic border 5506 around at least a portion of the right segment 1120 and may be activated to a different degree (i.e., indicated with an even brighter color, such as, for example, highlighter yellow) relative to the color, brightness, or other visual feature indicated by the other dynamic borders of the interactive menu 1110. When the top-right input device 1506 is actuated, the icons 1209C-D corresponding to the top-right input device 1506 may display a pre-defined background color, for example, that further highlights the icon 1209 corresponding to the top-right input device 1506, to indicate that the operation indicated by the icon 1209 is being performed. For example, the icon 1209 may display a yellow background color when the top-right input device 1506 is actuated. In some cases, the background color 1803 of the entire right segment 1120 may be set to a pre-defined background color 1803 when the top-right input device 1506 is actuated. For example, the right segment 1120 may change to have a transparent yellow background color 1803 when the top-right input device 1506 is actuated, and the transparency may provide readability to the text 1206 included in the right segment 1120.

In one embodiment, the top-right input device 1506 may correspond to the top operation icon 1209C while the bottom-right input device corresponds to the bottom operation icon 1209D. In this example, in response to the top-right input device 1506 being actuated, in addition to the background color being applied to the entire area of the of the right segment 1120 and its border 5506, the bottom operation icon 1209D may completely disappear. That is, the other operation is no longer available so it may no longer appear on the user interface. Similarly, in response to the bottom-right input device being actuated, the top operation icon 1209C may completely disappear or no longer appear on the user interface.

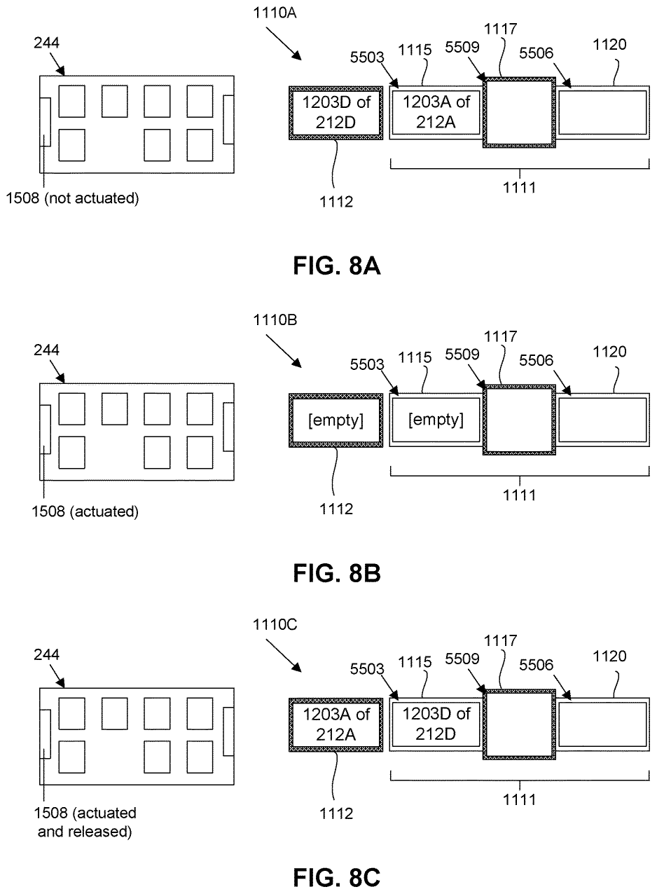

FIGS. 8A-8C illustrate various embodiments of the interactive menu 1110A-C based on whether the left instrument switch input device 1508 is actuated. As shown in FIGS. 8A-C, the dynamic border 5509 around the center segment 1117 is deactivated (shaded in grey), while the dynamic border 5503 around the left segment 1115, and the dynamic border 5506 around the right segment 1120 are activated (depicted in white). The shading of the dynamic borders 5503, 5506, and 5509 indicate the instruments 212 that are actively engaged to be controlled by the HIDs 226, 228. That is, the instruments 212 indicated in the left segment 1115 and the right segment 1120, with activated dynamic borders 5503, 5506, are actively controlled by the HIDs 226, 228. Meanwhile, the instrument 212 indicated in the center segment 1117, with the deactivated dynamic border 5509, are not actively controlled by the HIDs 226, 228.

FIGS. 8A-8C illustrate that, by actuating the left instrument switch input device 1508, the console 240 may receive an instrument control switch instruction. The instrument control switch instruction may be an instruction to switch control of a HID 226, 228 from a currently active instrument 212 (indicated in either the left segment 1115 or the right segment 1120) to an inactive instrument 212 (indicated in the inactive zone element 1112). In FIGS. 8A-8C, the instrument control switch instrument may trigger the console 240 to disengage the left HID 226 from controlling the robotic arm 210 coupled to the instrument 212A indicated in the left segment 1115 and engage the left HID 226 with the robotic arm 210 coupled to the instrument 212D indicated in the inactive zone element 1112. Specifically, FIG. 8A shows an embodiment of the interactive menu 1110A when the left instrument switch input device 1508 of the foot pedal assembly 244 is not actuated, FIG. 8B shows an embodiment of the interactive menu 1110B when or after the left instrument switch input device 1508 is actuated, and FIG. 8C shows an embodiment of the interactive menu 1110C after the left instrument switch input device 1508 is actuated and released.

FIG. 8A shows a first state of the interactive menu 1110A when the left segment 1115 indicates an active instrument 212A and the inactive zone element 1112 indicates an inactive instrument 212D. At the first state, which may be a first moment in time, the active instrument 212A may be coupled to an active robotic arm 210, and the inactive instrument 212D may be coupled to an inactive robotic arm 210. In this example, the left HID 226 may be assigned to control both the active robotic arm 210 and inactive robotic arm 210. The assignments may be preset by the system as a default assignment, or may be manipulated by the user using, for example, a user interface on a hand rest of the console 240.

FIG. 8B shows a second state of the interactive menu 1110B either when the left instrument switch input device 1508 is actuated, or shortly after the left instrument switch input device 1508 is actuated. In the second state, which may be a second moment in time after the first moment in time, the interactive menu 1110B may remove the instrument indicators 1203A describing instrument 212A from the left segment 1115 and the instrument indicators 1203D describing the inactive instrument 212D from the inactive zone element 1112.

FIG. 8C shows a third state of the interactive menu 1110C after the left instrument switch input device 1508 is actuated (and in some cases, released), and after the console 240 receives the instrument control switch instruction. In the third state, which may be a third moment in time after the second moment in time, the instrument 212D may become the active instrument 212, and the instrument 212A may become the inactive instrument 212A. The left segment 1115 may then indicate the instrument 212D and include the instrument indicators 1203D describing the instrument 212D. The inactive zone element 1112 may then indicate the instrument 212A and include the instrument indicators 1203A. In this state, the left HID 226 may be engaged with the active robotic arm 210 to control the instrument 212D.

It should be appreciated that different tool types may have different operations. For example, an energy tool may have a cut and a coagulation function whereas a non-energy tool may not have these operations. In other examples, a tool may have only one available operation, two operations, or no operations. In these examples, the operation icons 1209A-D described above may appear or disappear as the tools are swapped into or out of the left segment 1115 or the right segment 1120. In addition, even for a tool with one or more operations available, operation icons 1209A-D are not illustrated in response to the tool being deactivated and shown in the inactive zone element 1112. That is, in one example, the operation icons 1209A-D are only illustrated when the tools are selected and shown in the left segment 1115 or the right segment 1120. In one example, if a tool has only one available operation and it is in the left or right segment 1115, 1120, it may have only one operation icon 1209A illustrated.

### F. Status Indicators

In some embodiments, the interactive menu 1110 may include status indicators indicating a current status of a robotic arm 210 and/or an instrument 212 associated with the left segment 1115, center segment 1117, right segment 1120, and/or the inactive zone element 1112. The status indicators may be displayed in the interactive menu 1110 according to a status indicator priority.

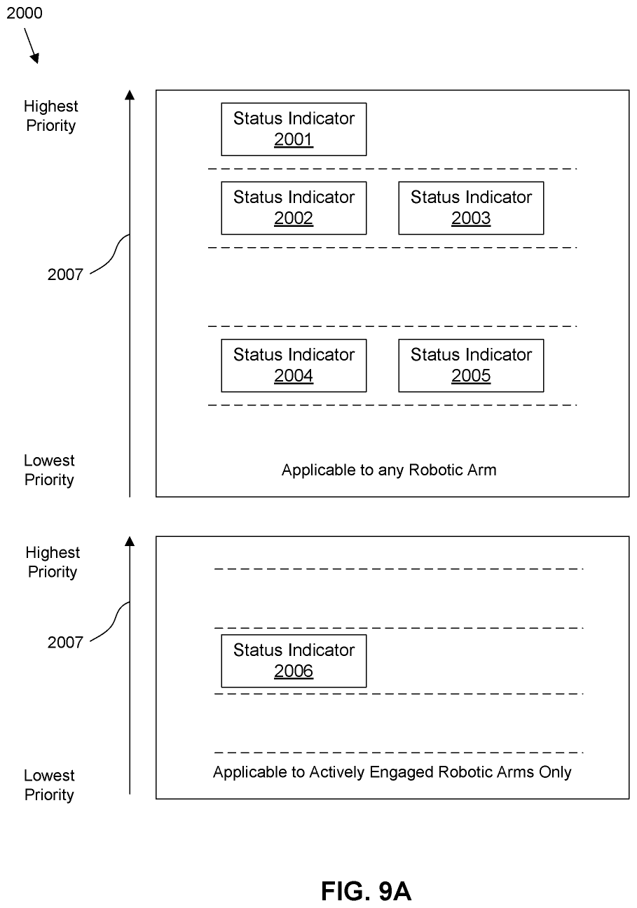

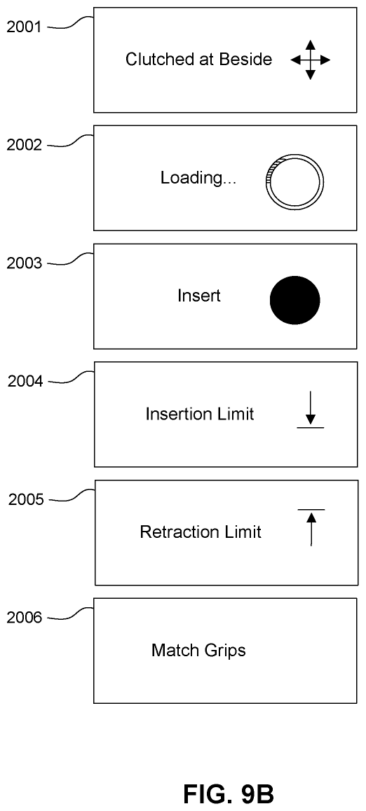

FIG. 9A depicts a chart 2000 illustrating various examples of status indicators 2001-2006 based on a status priority 2007 according to various embodiments of the disclosure. While FIG. 9 shows six different types of status indicators 2001-2006 as described, the interactive menu 1110 may include any number of status indicators 2001-2006. In an embodiment, the status indicators 2001-2006 may be adjusted to a higher priority or a lower priority, for example, by the user according to user input received at the console 240. For example, the status indicators 2001-2006 may be icons, and the chart 2000 may be displayed on a display (e.g., display device 242 or a touchscreen device) of the console 240. In this case, the user may drag and drop the status indicators 2001-2006 to different levels of priority within the chart 2000.

Each of the status indicators 2001-2006 may include text and/or icons, which when presented in the interactive menu 1110, may indicate a status of an instrument 212 indicated in a segment 1115, 1117, or 1120 proximate to the status indicator 2001-2007. For example, the status indicator 2001 may include text and/or an icon indicating clutched at bedside, which, when presented in the interactive menu 1110, may indicate that a robotic arm 210 coupled to an instrument 212 is clutched at bedside (e.g., by a patient support platform). The status indicator 2002 may include the text “Loading . . . ”, which, when presented in the interactive menu 1110, may indicate that a process of instrument loading on a robotic arm 210 has been initiated. The status indicator 2003 may include the text “Insert” and/or an icon including a downward arrow. This status indicator 2003, when presented in the interactive menu 1110, may indicate that an instrument 212 is being inserted into a robotic arm 210. The status indicator 2004 may include the text “Insertion Limit” and/or an icon including a downward arrow with a horizontal line. This status indicator 2004, when presented in the interactive menu 1110, may indicate that a particular instrument 212 has reached an insertion limit (i.e., cannot be inserted into the patient any further). The status indicator 2005 may include the text “Retraction Limit” and/or an icon including an upward arrow with a horizontal line. This status indictor 2005, when presented in the interactive menu 1110, may indicate that a particular instrument 212 has reached a retraction limit (i.e., cannot be retracted any further). The status indicator 2006 may include the text “Match Grips,” which, when presented in the interactive menu 1110, may indicate that the instrument 212 may need to be manually aligned and matched to a grip of the robotic arm 210. The status indicator 2006 may be a call to action to the medical provider immersed in the display device 242 or present in the room with the surgical robotic system, to perform the matching action prior to the HIDs 226, 228 being capable of controlling the robotic arm 210 and instrument 212.

In some embodiments, the status indicators 2001-2006 may be ordered according a status priority 2007 because only one status indicator 2001-2006 may be presented in the interactive menu 1110 for each segment 1115, 1117, 1120 and for the inactive zone element 1112 at once. In this way, when a robotic arm 210 or instrument 212 associated with a segment 1115, 1117, or 1120, or the inactive zone element 1112 has multiple status changes such that multiple status indicators 2001-2006 may apply, only one status indicator 2001-2006 may be presented for that segment 1115, 1117, or 1120, or the inactive zone element 1112. The displayed status indicator 2001-2006 may be determined based on the status priority 2007 shown in FIG. 9. For example, when the robotic arm 210 or the instrument 212 associated with the left segment 1115 has two status updates corresponding to status indicator 2001 and status indicator 2004, only one status indicator 2001 may be presented with the left segment 1115 (e.g., as an icon, text, or other indicator above the left segment 1115). This may be because status indicator 2001 has a higher status priority 2007 than the status indicator 2004.

FIG. 9B depicts examples of the status indicators 2001-2006, which may be displayed proximate to the segments 1115, 1117, and 1120 in the active zone element 1111. As shown in FIG. 9B, status indicator 2001 may be included with a segment 1115, 1117, and 1120 to indicate that the instrument 212A-B represented by the segment 1115, 1117, and 1120 is clutched at bedside. Status indicator 2002 may be included with a segment 1115, 1117, and 1120 to indicate that the instrument 212A-B represented by the segment 1115, 1117, and 1120 is being loaded onto a robotic arm 210. Status indicator 2003 may be included with a segment 1115, 1117, and 1120 to indicate that the instrument 212A-B represented by the segment 1115, 1117, and 1120 is being inserted into the robotic arm 210. Status indicator 2004 may be included with a segment 1115, 1117, and 1120 to indicate that the instrument 212A-B represented by the segment 1115, 1117, and 1120 has reached an insertion limit. Status indicator 2005 may be included with a segment 1115, 1117, and 1120 to indicate that the instrument 212A-B represented by the segment 1115, 1117, and 1120 has reached a retraction limit. Status indicator 2006 may be included with a segment 1115, 1117, and 1120 to indicate to the medical provider immersed in the display device 242 or present in the room with the surgical robotic system, to perform the matching action prior to the HIDs 226, 228 being capable of controlling the robotic arm 210 and instrument 212.

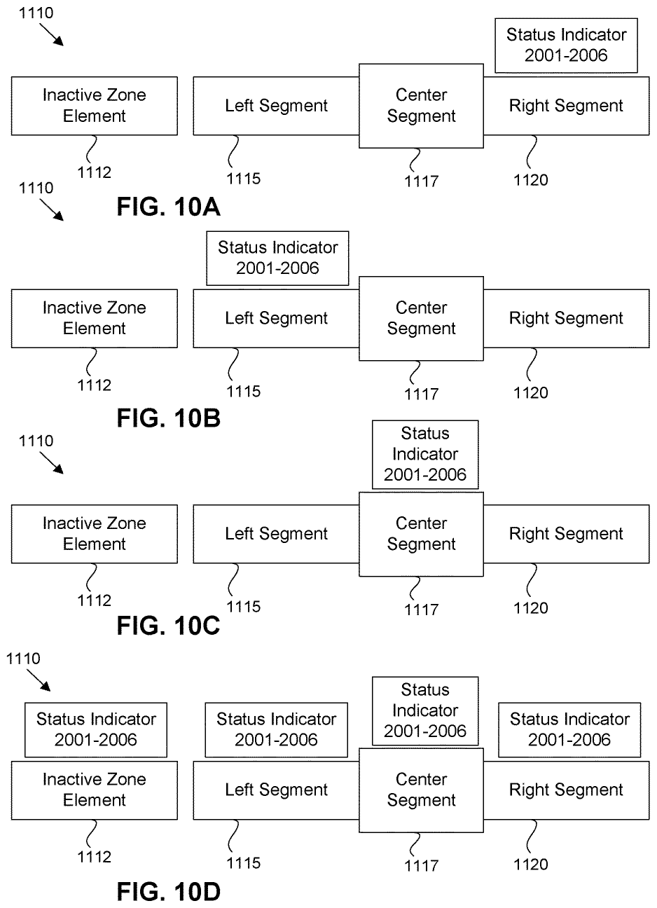

FIGS. 10A-10D illustrate examples of the positioning of one or more status indicators 2001-2006 in the interactive menu 1110 according to various embodiments of the disclosure. FIG. 10A illustrates the status indicators 2001-2006, positioned above and proximate to the right segment 1120, FIG. 10B illustrates the status indicators 2001-2006, positioned above and proximate to the left segment 1115, FIG. 10C illustrates the status indicators 2001-2006, positioned above and proximate to the center segment 1117, and FIG. 10D illustrates the status indicators 2001-2006, positioned above and proximate to the left segment 1115, center segment 1117, right segment 1120, and the inactive zone element 1112. FIG. 10D illustrates that one or more of the left segment 1115, center segment 1117, right segment 1120, and the inactive zone element 1112 may each have an associated status indicator 2001-2006. FIGS. 10A-10D indicate that only one status indicator 2001-2006 may be displayed at one time above each of the left segment 1115, center segment 1117, right segment 1120, and the inactive zone element 1112. Moreover, each of the left segment 1115, center segment 1117, right segment 1120, and the inactive zone element 1112 may each be displayed with one status indicator 2001-2006 at one time, as shown in FIG. 10D.

As shown in FIGS. 10A-10D, the status indicators 2001-2006 may be positioned proximate to the left segment 1115, center segment 1117, right segment 1120, and/or the inactive zone element 1112 describing an instrument 212 for which the status indicator 2001-2006 applies. In other words, the status indicator 2001-2006 may indicate a status of an instrument 212, and the status indicator 2001-2006 may be positioned in the interactive menu 1110 proximate to the segments 1115, 1117, 1120, and/or the inactive zone element 1112 describing the instrument 212.

While FIGS. 10A-10D show the status indicators 2001-2006 as being positioned above the left segment 1115, center segment 1117, right segment 1120, and/or the inactive zone element 1112, it should be appreciated that the status indicators 2001-2006 may be otherwise positioned anywhere proximate to the left segment 1115, center segment 1117, right segment 1120, and/or the inactive zone element 1112. For example, the status indicators 2001-2006 may be positioned below the left segment 1115, center segment 1117, right segment 1120, and/or the inactive zone element 1112, the status indicators 2001-2006 may be positioned adjacent to the left segment 1115, center segment 1117, right segment 1120, and/or the inactive zone element 1112, etc.

### G. Table Indicator

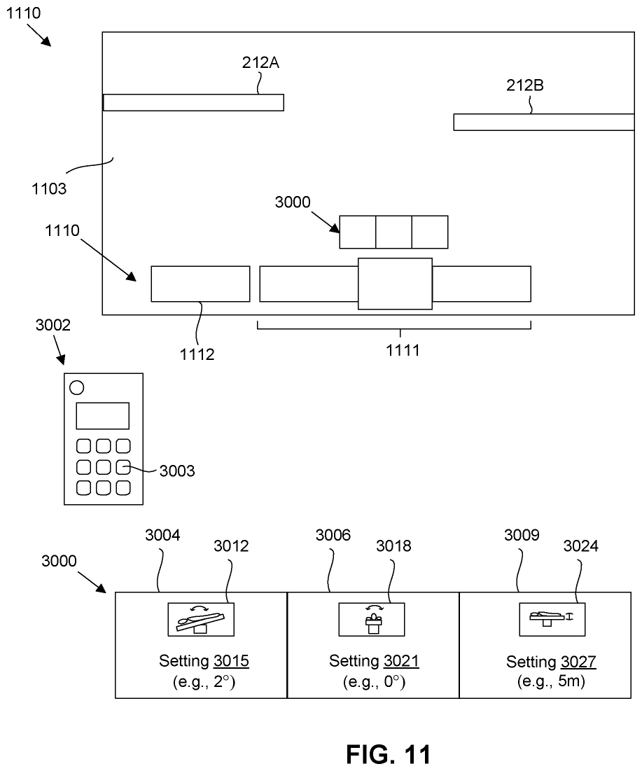

In an embodiment in which the surgical robotic system includes a patient platform or table upon which a patient rests during a procedure, the display device 242 may additionally display a table indicator with the interactive menu 1110. FIG. 11 illustrates a table indicator 3000 displayed at the display device 242 above the interactive menu 1110 according to various embodiments of the disclosure. Similar to the interactive menu 1110, the table indicator 3000 may be overlaid on the image 1103 displayed at the display device 242.

In an embodiment, the table indicator 3000 may generally display information related to the types of adjustments that may be made to the orientation and/or height of the patient table. In the example shown in FIG. 11, the table indicator 3000 includes three components 3004, 3006, and 3009, which may each correspond to different types of adjustments that may be made to the patient table. For example, the component 3004 may describe a tilt adjustment of the patient table. A tilt adjustment may refer to the adjustment of an angle of the patient table in a vertical direction along a length of the table. For example, a tilt adjustment may be made to the patient table in the head-up (head-elevated) orientation or the feet-up (feet-elevated) orientation. A tilt adjustment may also refer to the adjustment of an angle of the patient table in a horizontal direction along a width of the table. For example, a tilt adjustment may be made to the patient table in a left-side elevated orientation or a right-side elevated orientation. The component 3006 may describe a trend adjustment of the patient table. A trend adjustment may refer to the longitudinal or latitudinal movement of the patient table. For example, the trend adjustment may involve sliding the patient table back and forth in a linear direction, along the length of the patient and/or along the width of the patient. The component 3009 may describe a height adjustment of the patient table. For example, the height adjustment may involve sliding the patient table up and down in a linear, vertical direction.

While the table indicator 3000 shown in FIG. 11 only includes three components 3004, 3006, and 3009, it should be appreciated that the table indicator 3000 may include any number of components, each corresponding to a different type of adjustment that may be made to the patient table. In some embodiments, there may two components 3004 describing different tilt adjustments. For example, there may be a first component 3004 describing a tilt adjustment to the patient table along a length of the table (i.e., head-elevated or feet-elevated), and a second component 3004 describing a tilt adjustment to the patient table along a width of the table (i.e., left-side elevated or right-side elevated).

Each of the components 3004, 3006, and 3009 may display text, icons, images, and/or other user interface components identifying a type of and/or describing a setting associated with the adjustment. The component 3004 describing the tilt adjustment may include a graphical representation 3012 of the patient table, for example, depicting a patient table and a patient positioned on the table. The graphical representation 3012 of the patient table may be titled to match (i.e., be proportional) the tilt of the actual patient table. The graphical representation 3012 of the patient table may be oriented in either a length direction or a width direction of the patient to indicate a direction of the tilt being indicated in the component 3004. For example, when the tilt adjustment is along the length of the table, the graphical representation 3012 of the patient table may be oriented to depict a length of the patient. When the tilt adjustment is along the width of the table, the graphical representation 3012 of the patient table may be oriented to depict a width of the patient. The graphical representation 3012 of the patient table may also include an arched, double sided arrow positioned horizontally above the patient table, indicating a direction of the tilt adjustment described in the component 3004. The component 3004 may also include text describing a setting 3015 associated with the tilt adjustment. For example, the text describing the setting 3015 may recite an angle (in degrees) by which the patient table is tilted.

The component 3006 may similarly include a graphical representation 3018 of the patient table, in which a trend (i.e., relative translation) of the graphical representation 3015 of the patient table is proportional to a trend of the actual patient table. The graphical representation 3018 of the patient table may be oriented in either a length direction or a width direction of the patient to indicate a direction of the trend being indicated in the component 3006. The graphical representation 3018 of the patient table may also include a double-sided arrow positioned above the patient table in the horizontal direction, indicating a direction of the trend adjustment described in the component 3006. The component 3006 may also include text describing a setting 3021 associated with the trend adjustment. For example, the text describing the setting 3021 may recite a distance by which the patient table is translated (or trended) longitudinally or latitudinally relative to a start/default position.

The component 3009 may similarly include a graphical representation 3024 of the patient table, in which a height adjustment of the graphical representation 3015 of the patient table is proportional to a relative height of the actual patient table. The graphical representation 3023 of the patient table may be oriented in either a length direction or a width direction of the patient. The graphical representation 3024 of the patient table may also include a double-sided arrow positioned on the side of the patient table in a vertical direction, indicating a direction of the height adjustment described in the component 3009. The component 3009 may also include text describing a setting 3027 associated with the height adjustment. For example, the height describing the setting 2037 may recite a height by which the patient table is translated (or trended) vertically relative to a start/default position.

While FIG. 11 shows the table indicator 3000 as being positioned directly above the interactive menu 1110, it should be appreciated that the table indicator 3000 may be positioned anywhere on the display device 242. FIG. 11 depicts the size of the table indicator 3000 as being considerably smaller than the size of the interactive menu 1110. However, it should be appreciated that the size of the table indicator 3000 may vary in different embodiments to be the same size or comparable in size to the interactive menu 1110, or larger in size than the interactive menu 1110. The positioning and the size of the table indicator 3000 may vary so long as the positioning and size of the table indicator 3000 does not obstruct the view of the patient anatomy interacting with the instruments 212A-B as displayed at the display device 242.

In an embodiment, the table indicator 3000 may be displayed at all times or only in response to a user input received at one or more input devices of the robotic system, in which the input devices may be positioned at the console 240 or another device operating to control the robotic system. For example, the device may be a remote control 3002, including, for example, one or more input devices 3003, some of which may be used to adjust the patient table as described herein. For example, one or more first input devices 3003 at the remote control 3002 may control a tilt adjustment in a vertical direction, one or more second input devices 3003 at the remote control 3002 may control a tilt adjustment in a horizontal direction, one or more third input devices 3003 at the remote control 3002 may control a trend adjustment, and one or more fourth input devices 3003 at the remote control 3002 may control a height adjustment. The user may select or otherwise interact with the input devices 3002 accordingly to control the adjustments to the patient table.

In an embodiment, the table indicator 3000 may only be displayed when the input devices 3003 are being used to adjust the settings of the patient table. The information in the components 3004, 3006, and/or 3009 may adjust in real-time as the settings of the actual patient table are also being adjusted. For example, when the input device 3003 is selected to change the tilt of the patient table from the head-elevated orientation to the feet-elevated orientation, the graphical representation 3012 of the patient table may similarly update to depict the feet-elevated orientation. Similarly, the text describing the setting 3015, which may recite an angle of the table, may also be updated accordingly.

In an embodiment, the table indicator 3000 may stop being displayed at the display device 242 when the input device 3003 is no longer being manipulated, selected, or otherwise interacted with to adjust the patient table settings. In an embodiment, the table indicator 3000 may be displayed for a predetermined period of time after the patient table setting has been adjusted or after the input device 3003 is no longer being used to adjust the setting of the table. The predetermined period of time may be, for example, between 2 and 5 seconds, or may be 3 seconds. In this way, the table indicator 3000 may only be displayed when the patient table settings may be relevant to the medical provider setting up the robotic system or performing a procedure on the patient. This may in turn prevent the medical provider from being distracted by the table indicator 3000 for an unnecessarily long period of time.

### H. Exemplary Processes

FIG. 12 is a flowchart illustrating method 2200 performed by the surgical robotic system. Specifically, method 2200 may be performed by a processor in the console 240 or coupled to the console 240.

At step 2203, method 2200 comprises displaying, at a console 240 of the surgical robotic system, an interactive menu 1110 comprising an active zone element 1111 and an inactive zone element 1112. In an embodiment, the active zone element 1111 comprises a left segment 1115, a right segment 1120, and a center segment 1117.

At step 2206, method 2200 comprises indicating, in the left segment 1115 of the active zone element 1111, a first instrument 212 coupled to a first robotic arm 210 of the surgical robotic system and controlled by a left HID 226 of the console 240. At step 2209, method 2200 comprises indicating, in the right segment 1120 of the active zone element 1111, a second instrument 212 coupled to a second robotic arm 210 of the surgical robotic system and controlled by a right HID 228 of the console 240. At step 2212, method 2200 comprises indicating, in the center segment 1117 of the active zone element 1111, data associated with a camera coupled to a third robotic arm 210 of the surgical robotic system. At step 2215, method 2200 comprises indicating, in the inactive zone element 1112, a third instrument 212 coupled to a fourth robotic arm 210. In an embodiment, the right HID 228 may be engaged to control the fourth robotic arm 210 when an instruction is received to switch control of the right HID 228 from the second instrument 212 to the third instrument 212.

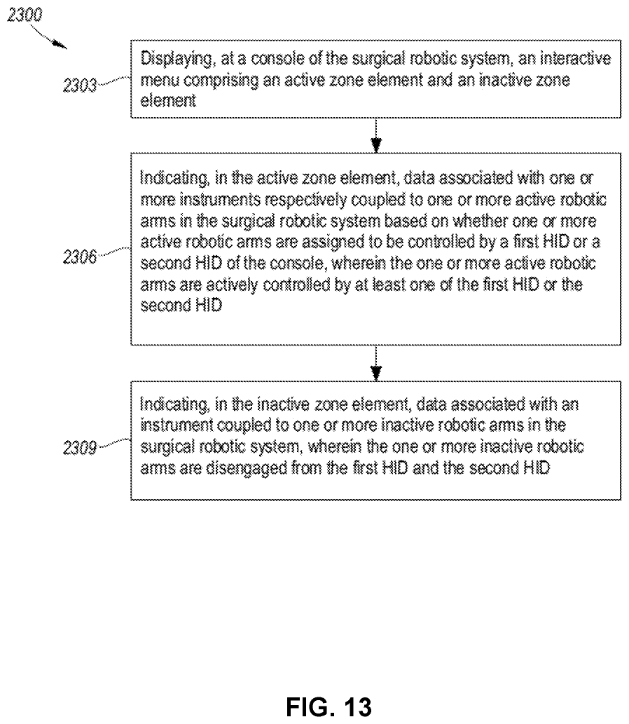

FIG. 13 is a flowchart illustrating method 2300 performed by the surgical robotic system. Specifically, method 2300 may be performed by a processor in the console 240 or coupled to the console 240.

At step 2303, method 2300 comprises displaying, at a console 240 of the surgical robotic system, an interactive menu 1110 comprising an active zone element 1111 and an inactive zone element 1112. At step 2306, method 2300 comprises indicating, in the active zone element 1111, data associated with one or more instruments 212 respectively coupled to one or more active robotic arms 210 in the surgical robotic system based on whether one or more active robotic arms 210 are assigned to be controlled by a first HID 226 or a second HID 228 of the console 240. In an embodiment, the one or more active robotic arms 210 are actively controlled by at least one of the first HID 226 or the second HID 228. At step 2309, method 2300 comprises indicating, in the inactive zone element 1112, data associated with an instrument 212 coupled to one or more inactive robotic arms 210 in the surgical robotic system. The one or more inactive robotic arms 210 are disengaged from the first HID 226 and the second HID 228.

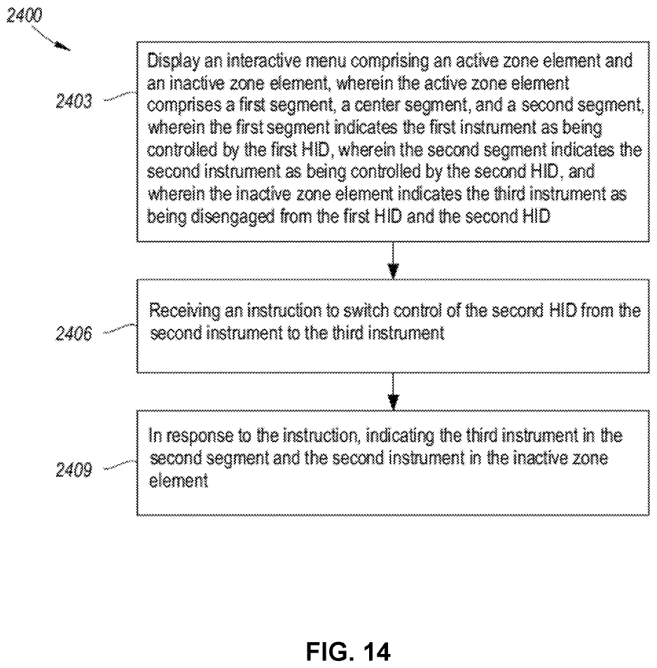

FIG. 14 is a flowchart illustrating method 2400 performed by the surgical robotic system. Specifically, method 2400 may be performed by a processor in the console 240 or coupled to the console 240.

At step 2403, method 2400 comprises displaying an interactive menu 1110 comprising an active zone element 1111 and an inactive zone element 1112, in which the active zone element may comprise a first segment 1115, a center segment 1117, and a second segment 1120. The first segment 1115 may indicate the first instrument 212 as being controlled by the first HID 226, and the second segment 1120 indicates the second instrument 212 as being controlled by the second HID 228. The inactive zone element 1112 may indicate the third instrument 212 as being disengaged from the first HID 226 and the second HID 228. At step 2406, method 2400 comprises receiving an instruction to switch control of the second HID 228 from the second instrument 212 to the third instrument 212. At step 2409, method 2400 comprises in response to receiving the instruction, indicating the third instrument 212 in the second segment 1120 and the second instrument 212 in the inactive zone element 1112.

### I. Implementing Systems and Terminology

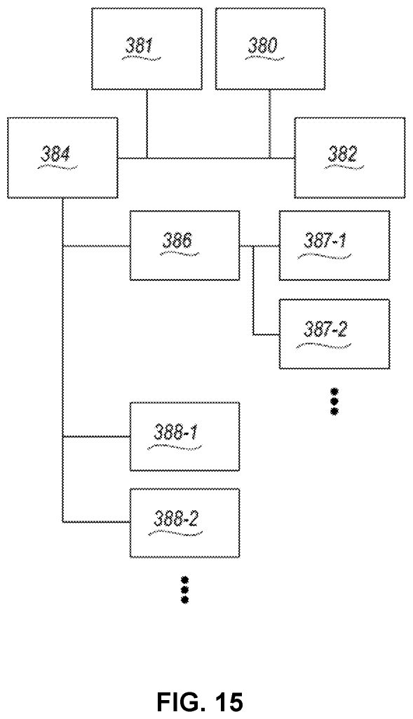

FIG. 15 is a schematic diagram illustrating electronic components of a surgical robotic system in accordance with some embodiments.

The surgical robotic system includes one or more processors 380, which are in communication with a computer-readable storage medium 382 (e.g., computer memory devices, such as random-access memory, read-only memory, static random-access memory, and non-volatile memory, and other storage devices, such as a hard drive, an optical disk, a magnetic tape recording, or any combination thereof) storing instructions for performing any methods described herein (e.g., operations described with respect to FIGS. 1, 2, 3, 4, 5, 6A, 6B, 7, 8A, 8B, 8C, 9, 10A, 10B, 10C, 10D, 11, 12, and 13). The one or more processors 380 are also in communication with an input/output controller 384 (via a system bus or any suitable electrical circuit). The input/output controller 384 receives sensor data from one or more sensors 388-1, 388-2, etc., and relays the sensor data to the one or more processors 380. The input/output controller 384 also receives instructions and/or data from the one or more processors 380 and relays the instructions and/or data to one or more actuators, such as first motors 387-1 and 387-2, etc. In some embodiments, the input/output controller 384 is coupled to one or more actuator controllers 386 and provides instructions and/or data to at least a subset of the one or more actuator controllers 386, which, in turn, provide control signals to selected actuators. In some embodiments, the one or more actuator controllers 386 are integrated with the input/output controller 384 and the input/output controller 384 provides control signals directly to the one or more motors 387-1 and 387-2, etc. (without a separate actuator controller). Although FIG. 15 shows that there is one actuator controller 386 (e.g., one actuator controller for the entire surgical robotic system; in some embodiments, additional actuator controllers may be used (e.g., one actuator controller for each actuator, etc.)). In some embodiments, the one or more processors 380 are in communication with one or more displays 381 for displaying information as described herein.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The functions for determining whether a tool is within or outside a surgical field of view provided by a camera or scope and rendering one or more indicators representing positions or directions of one or more medical tools described herein may be stored as one or more instructions on a processor-readable or computer-readable medium. The term “computer-readable medium” refers to any available medium that can be accessed by a computer or processor. By way of example, and not limitation, such a medium may comprise random access memory (RAM), read-only memory (ROM), electrically erasable programmable read-only memory (EEPROM), flash memory, compact disc read-only memory (CD-ROM) or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

As used herein, the term “exemplary” means “serving as an example, instance, or illustration,” and does not necessarily indicate any preference or superiority of the example over any other configurations or implementations.

As used herein, the term “and/or” encompasses any combination of listed elements. For example, “A, B, and/or C” includes the following sets of elements: A only, B only, C only, A and B without C, A and C without B, B and C without A, and a combination of all three elements, A, B, and C.

1. A surgical robotic system, comprising:
a plurality of robotic arms comprising an active robotic arm and an inactive robotic arm;
a console communicatively coupled to the robotic arms, comprising:
a first haptic interface device (HID) and a second HID; and
a display configured to:
display an interactive menu comprising an active zone element and an inactive zone element;
indicate, in the active zone element, data associated with a first instrument coupled to the active robotic arm based on whether the active robotic arm is assigned to be controlled by the first HID or the second HID; and
indicate, in the inactive zone element, data associated with a second instrument coupled to the inactive robotic arm, wherein the inactive robotic arm is disengaged from the first HID and the second HID.

2. The surgical robotic system of claim 1, wherein the plurality of robotic arms comprise a first active robotic arm, a second active robotic arm, and a third active robotic arm, wherein the active zone element comprises a first segment, a second segment, and a center segment, wherein to indicate, in the active zone element, the data associated with the first instrument and the data associated with the second instrument, the display is further configured to:
indicate, in the first segment of the active zone element, the first instrument coupled to the first active robotic arm and controlled by the first HID;
indicate, in the second segment of the active zone element, a third instrument coupled to the second active robotic arm and controlled by the second HID; and
indicate, in the center segment of the active zone element, data associated with a camera coupled to the third active robotic arm.

3. The surgical robotic system of claim 2, wherein the display is further configured to indicate, in the inactive zone element, the second instrument coupled to an inactive robotic arm, wherein the inactive robotic arm is assigned to be controlled by the second HID when an instruction is received to switch control of the second HID from the third instrument to the second instrument, and wherein the inactive zone element is positioned proximate to the second segment of the active zone element.

4. The surgical robotic system of claim 3, wherein the console further comprises a processor coupled to the display, wherein the processor is configured to:
receive the instruction to switch control of the second HID from the third instrument to the second instrument;
wherein, in response to the instruction being received, the display being configured to:
replace, in the second segment of the active zone element, an indication of the third instrument with an indication of the second instrument; and
replace, in the inactive zone element, the indication of the second instrument with the indication of the third instrument.

5. The surgical robotic system of claim 2, wherein the data associated with the camera comprises at least one an orientation of the camera, a type of the camera, or an identifier of the third active robotic arm to which the camera is coupled.

6. The surgical robotic system of claim 2, wherein the data associated with the first instrument or the data associated with the second instrument comprises at least one of an identification of an instrument, one or more operations to be performed using the instrument, a background color indicating a status of the one or more operations, an operation indication color identifying the one or more operations, an identifier of a robotic arm to which the instrument is coupled, or a color identifying whether the instrument is engaged by the first HID or the second HID.

7. The surgical robotic system of claim 1, wherein the active robotic arm is coupled to a camera configured to capture an image, and wherein the interactive menu is overlaid on the image.

8. The surgical robotic system of claim 7, wherein the camera is an endoscopic device or a laparoscopic device.

9. The surgical robotic system of claim 1, wherein the interactive menu is overlaid over a portion of an image, wherein a size of the interactive menu is less than a pre-defined percentage of a size of the image, and wherein the interactive menu is positioned proximate to an edge of the image.

10. The surgical robotic system of claim 1, wherein the inactive zone element comprises at least one of an identification of the second instrument or an identifier of the inactive robotic arm to which the second instrument is coupled.

11. The surgical robotic system of claim 1, wherein the console comprises a plurality of foot actuators, wherein each of the foot actuators, when actuated, activates at least one of:
an instrument mode to perform an operation using the first instrument,
a clutch mode to disengage the first instrument from the active robotic arm,
a scope mode to control movement of a third robotic arm coupled to a camera,
an instrument switch mode to lock the active robotic arm while an instrument switch is manually performed at the active robotic arm, or
a switch instruction to switch control of the second HID from an active instrument to the second instrument.

12. The surgical robotic system of claim 1, wherein the display is further configured to indicate, proximate to the active zone element or the inactive zone element, one or more status indicators according to a status indicator priority, wherein the one or more status indicators indicate a current status of at least one of a robotic arm or an instrument.

13. A method performed by a surgical robotic system, comprising:
displaying, at a console of the surgical robotic system, an interactive menu comprising an active zone element and an inactive zone element, wherein the active zone element comprises a left segment, a center segment, and a right segment;
indicating, in the left segment of the active zone element, a first instrument coupled to a first robotic arm of the surgical robotic system and controlled by a left haptic interface device (HID) of the console;
indicating, in the right segment of the active zone element, a second instrument coupled to a second robotic arm of the surgical robotic system and controlled by a right HID of the console;
indicating, in the center segment of the active zone element, data associated with a camera coupled to a third robotic arm of the surgical robotic system; and
indicating, in the inactive zone element, a third instrument coupled to a fourth robotic arm, wherein the right HID is engaged to control the fourth robotic arm when an instruction is received to switch control of the right HID from the second instrument to the third instrument.

14. (canceled)

15. The method of claim 13, wherein the data associated with the camera comprises at least one of an orientation of the camera, a type of the camera, or an identifier of the fourth robotic arm to which the camera is coupled.

16. The method of claim 13, wherein the left segment further indicates at least one of an identification of the first instrument, one or more operations to be performed using the first instrument, a background color indicating a status of the one or more operations, an operation indication color identifying the one or more operations, an identifier of the first robotic arm to which the first instrument is coupled, or a color identifying whether the first instrument is engaged by the left HID.

17. (canceled)

18. The method of claim 13, wherein the interactive menu is overlaid over a portion of an image obtained by the camera, wherein a size of the interactive menu is less than a pre-defined percentage of a size of the image, and wherein the interactive menu is positioned proximate to an edge of the image.

19. The method of claim 13, further comprising:
receiving the instruction to switch control of the right HID from the second instrument to the third instrument;
after receiving the instruction, the method further comprises:
replacing, in the right segment of the active zone element, an indication of the second instrument with an indication of the third instrument; and
replacing, in the inactive zone element, the indication of the third instrument with the indication of the second instrument.

20. The method of claim 13, wherein the inactive zone element comprises at least one of an identification of the third instrument or an identifier of the third robotic arm to which the third instrument is coupled.

21. The method of claim 13, wherein the console comprises a plurality of foot actuators, wherein each of the foot actuators, when actuated, activates at least one of:
an instrument mode to perform an operation using one or more instruments respectively coupled to a robotic arm,
a clutch mode to perform a clutching operation using the one or more instruments respectively coupled to the first robotic arm to the second robotic arm,
a scope mode to control movement of a robotic arm coupled to a camera,
an instrument switch mode to lock one or more active robotic arms while an instrument switch is manually performed at one of the robotic arms, or
a switch instruction to switch control of the right HID from the second instrument to the third instrument.

22. (canceled)

23. (canceled)

24. A non-transitory, computer-readable medium storing instructions which, when executed by a processor of a surgical robotic system, cause the processor to:
display, at a console of the surgical robotic system, an interactive menu comprising an active zone element and an inactive zone element;
indicate, in the active zone element, data associated with one or more instruments respectively coupled to one or more active robotic arms in the surgical robotic system based on whether one or more active robotic arms are assigned to be controlled by a first haptic interface device (HID) or a second HID of the console, wherein the one or more active robotic arms are actively controlled by at least one of the first HID or the second HID; and
indicate, in the inactive zone element, data associated with an instrument coupled to one or more inactive robotic arms in the surgical robotic system, wherein the one or more inactive robotic arms are disengaged from the first HID and the second HID.
